SEQUENCE LISTING (94 sequences)

SEQ ID NO: 1 - DNA, 20 bp, synthetic construct
  source 1..20: mol_type = other DNA
cacatctgcccaagccaagg

SEQ ID NO: 2 - DNA, 20 bp, synthetic construct
  source 1..20: mol_type = other DNA
ctgttggcctccagaagcac

SEQ ID NO: 3 - DNA, 20 bp, synthetic construct
  source 1..20: mol_type = other DNA
ctgttcccccggttaccctc

SEQ ID NO: 4 - DNA, 19 bp, synthetic construct
  source 1..19: mol_type = other DNA
gtccctgggccttggtttg

SEQ ID NO: 5 - DNA, 22 bp, synthetic construct
  source 1..22: mol_type = other DNA
ctgcatcataattccagcagga

SEQ ID NO: 6 - DNA, 23 bp, synthetic construct
  source 1..23: mol_type = other DNA
ctctcgctttattcttccttttc

SEQ ID NO: 7 - DNA, 21 bp, synthetic construct
  source 1..21: mol_type = other DNA
ggataacttccctatcagata

SEQ ID NO: 8 - DNA, 20 bp, synthetic construct
  source 1..20: mol_type = other DNA
gtgtctatcctttaccacag

SEQ ID NO: 9 - DNA, 25 bp, synthetic construct
  source 1..25: mol_type = other DNA
agaactggactacaaatgcaggagt

SEQ ID NO: 10 - DNA, 22 bp, synthetic construct
  source 1..22: mol_type = other DNA
ggggctatcactcctgttcctc

SEQ ID NO: 11 - DNA, 22 bp, synthetic construct
  source 1..22: mol_type = other DNA
aggctgggcatggtggctcata

SEQ ID NO: 12 - DNA, 21 bp, synthetic construct
  source 1..21: mol_type = other DNA
cgcgcccggccttaattttgt

SEQ ID NO: 13 - DNA, 22 bp, synthetic construct
  source 1..22: mol_type = other DNA
ggacagtggagacagatagtct

SEQ ID NO: 14 - DNA, 21 bp, synthetic construct
  source 1..21: mol_type = other DNA
ccttcaggcctagaacgaaac

SEQ ID NO: 15 - DNA, 20 bp, synthetic construct
  source 1..20: mol_type = other DNA
tcagtgccagtgacctgttg

SEQ ID NO: 16 - DNA, 20 bp, synthetic construct
  source 1..20: mol_type = other DNA
tgtcagagcaagggcctatc

SEQ ID NO: 17 - DNA, 23 bp, synthetic construct
  source 1..23: mol_type = other DNA
tcacttttcatgagcagggatgc

SEQ ID NO: 18 - DNA, 23 bp, synthetic construct
  source 1..23: mol_type = other DNA
aaacaaacttggctctgggtagg

SEQ ID NO: 19 - DNA, 22 bp, synthetic construct
  source 1..22: mol_type = other DNA
ctgcgggcctgggccgcactga

SEQ ID NO: 20 - DNA, 20 bp, synthetic construct
  source 1..20: mol_type = other DNA
gcgggcaggaggaacggcta

SEQ ID NO: 21 - DNA, 20 bp, synthetic construct
  source 1..20: mol_type = other DNA
gctggtcggagctacttcct

SEQ ID NO: 22 - DNA, 20 bp, synthetic construct
  source 1..20: mol_type = other DNA
tcatttcctgggggtctggc

SEQ ID NO: 23 - DNA, 23 bp, synthetic construct
  source 1..23: mol_type = other DNA
gctttttgcgtcctggtgttgat

SEQ ID NO: 24 - DNA, 18 bp, synthetic construct
  source 1..18: mol_type = other DNA
gcgagtgcgagccgtgag

SEQ ID NO: 25 - DNA, 22 bp, synthetic construct
  source 1..22: mol_type = other DNA
cgggcctgggccctcggcccca

SEQ ID NO: 26 - DNA, 19 bp, synthetic construct
  source 1..19: mol_type = other DNA
aggggcaagaagcatggcc

SEQ ID NO: 27 - DNA, 20 bp, synthetic construct
  source 1..20: mol_type = other DNA
gggctgtcaagatcaggcgt

SEQ ID NO: 28 - DNA, 20 bp, synthetic construct
  source 1..20: mol_type = other DNA
ggtgacctgggggtgaaagt

SEQ ID NO: 29 - DNA, 21 bp, synthetic construct
  source 1..21: mol_type = other DNA
tcactgccctgaagaaacacc

SEQ ID NO: 30 - DNA, 21 bp, synthetic construct
  source 1..21: mol_type = other DNA
ccgcgcccggccatgtacatt

SEQ ID NO: 31 - DNA, 21 bp, synthetic construct
  source 1..21: mol_type = other DNA
aggtcaggacgcgagaggaag

SEQ ID NO: 32 - DNA, 25 bp, synthetic construct
  source 1..25: mol_type = other DNA
tgggcagagtcggccgtcctcgcag

SEQ ID NO: 33 - DNA, 25 bp, synthetic construct
  source 1..25: mol_type = other DNA
cctcttggatccacgttctagtttc

SEQ ID NO: 34 - DNA, 26 bp, synthetic construct
  source 1..26: mol_type = other DNA
cttcagtgatgctccagatgtaatcc

SEQ ID NO: 35 - DNA, 19 bp, synthetic construct
  source 1..19: mol_type = other DNA
gataggggtcctcggcctg

SEQ ID NO: 36 - DNA, 20 bp, synthetic construct
  source 1..20: mol_type = other DNA
ctgtttggtgggtgctgtcg

SEQ ID NO: 37 - DNA, 20 bp, synthetic construct
  source 1..20: mol_type = other DNA
cggaggccaaccagacttgc

SEQ ID NO: 38 - DNA, 22 bp, synthetic construct
  source 1..22: mol_type = other DNA
agtggctgagacttgtgtcctg

SEQ ID NO: 39 - DNA, 27 bp, synthetic construct
  source 1..27: mol_type = other DNA
cttgccaggaacttgtccagggagtcg

SEQ ID NO: 40 - DNA, 32 bp, synthetic construct
  source 1..32: mol_type = other DNA
cttgtgtgtcaggaacttgtccagggaggact

SEQ ID NO: 41 - DNA, 22 bp, synthetic construct
  source 1..22: mol_type = other DNA
cggctaccgaggctccagcgta

SEQ ID NO: 42 - DNA, 27 bp, synthetic construct
  source 1..27: mol_type = other DNA
gtgtcggctaccgaggctccagctgga

SEQ ID NO: 43 - DNA, 25 bp, synthetic construct
  source 1..25: mol_type = other DNA
ctcacagaagccaggaacttgtaca

SEQ ID NO: 44 - DNA, 26 bp, synthetic construct
  source 1..26: mol_type = other DNA
ctcacagaagccaggaacttgtaagg

SEQ ID NO: 45 - DNA, 23 bp, synthetic construct
  source 1..23: mol_type = other DNA
caaaggaggatgtttttagtagc

SEQ ID NO: 46 - DNA, 20 bp, synthetic construct
  source 1..20: mol_type = other DNA
gctctgccctgacttttctg

SEQ ID NO: 47 - DNA, 21 bp, synthetic construct
  source 1..21: mol_type = other DNA
gctctgccctgacttttagtc

SEQ ID NO: 48 - DNA, 22 bp, synthetic construct
  source 1..22: mol_type = other DNA
gatggctctgccctgacttgta

SEQ ID NO: 49 - DNA, 23 bp, synthetic construct
  source 1..23: mol_type = other DNA
gatggctctgccctgactttggt

SEQ ID NO: 50 - DNA, 25 bp, synthetic construct
  source 1..25: mol_type = other DNA
aatagatggctctgccctgactgtt

SEQ ID NO: 51 - DNA, 19 bp, synthetic construct
  source 1..19: mol_type = other DNA
tggctctgccctgacttac

SEQ ID NO: 52 - DNA, 26 bp, synthetic construct
  source 1..26: mol_type = other DNA
gcaatagatggctctgccctgacgtt

SEQ ID NO: 53 - DNA, 27 bp, synthetic construct
  source 1..27: mol_type = other DNA
gcaatagatggctctgccctgactgct

SEQ ID NO: 54 - DNA, 24 bp, synthetic construct
  source 1..24: mol_type = other DNA
cacagttgtgtcagaagcaaaggt

SEQ ID NO: 55 - DNA, 23 bp, synthetic construct
  source 1..23: mol_type = other DNA
cagttgtgtcagaagcaaattga

SEQ ID NO: 56 - DNA, 23 bp, synthetic construct
  source 1..23: mol_type = other DNA
ctcctcaggagtcagatgcaaca

SEQ ID NO: 57 - DNA, 24 bp, synthetic construct
  source 1..24: mol_type = other DNA
ctcctcaggagtcagatgcacact

SEQ ID NO: 58 - DNA, 19 bp, synthetic construct
  source 1..19: mol_type = other DNA
acgttcaccttgcccccca

SEQ ID NO: 59 - DNA, 20 bp, synthetic construct
  source 1..20: mol_type = other DNA
acgttcaccttgccccaaca

SEQ ID NO: 60 - DNA, 22 bp, synthetic construct
  source 1..22: mol_type = other DNA
ccaacttcatccacgttcaact

SEQ ID NO: 61 - DNA, 23 bp, synthetic construct
  source 1..23: mol_type = other DNA
ccaacttcatccacgttcacaat

SEQ ID NO: 62 - DNA, 20 bp, synthetic construct
  source 1..20: mol_type = other DNA
accaacctgcccagggcatc

SEQ ID NO: 63 - DNA, 21 bp, synthetic construct
  source 1..21: mol_type = other DNA
accaacctgcccagggccgta

SEQ ID NO: 64 - DNA, 20 bp, synthetic construct
  source 1..20: mol_type = other DNA
gataccaacctgcccatggc

SEQ ID NO: 65 - DNA, 21 bp, synthetic construct
  source 1..21: mol_type = other DNA
gataccaacctgcccaggtgc

SEQ ID NO: 66 - DNA, 20 bp, synthetic construct
  source 1..20: mol_type = other DNA
cttgtaaccttgataccgac

SEQ ID NO: 67 - DNA, 21 bp, synthetic construct
  source 1..21: mol_type = other DNA
cttgtaaccttgataccacac

SEQ ID NO: 68 - DNA, 21 bp, synthetic construct
  source 1..21: mol_type = other DNA
cttgtaaccttgataccactc

SEQ ID NO: 69 - DNA, 29 bp, synthetic construct
  source 1..29: mol_type = other DNA
tccttaaacctgtcttgtaaccttgagac

SEQ ID NO: 70 - DNA, 29 bp, synthetic construct
  source 1..29: mol_type = other DNA
tccttaaacctgtcttgtaaccttgatcg

SEQ ID NO: 71 - DNA, 22 bp, synthetic construct
  source 1..22: mol_type = other DNA
tcctgagacttccacactgatg

SEQ ID NO: 72 - DNA, 23 bp, synthetic construct
  source 1..23: mol_type = other DNA
aagaaagtgctcggtgcctttcg

SEQ ID NO: 73 - DNA, 24 bp, synthetic construct
  source 1..24: mol_type = other DNA
aagaaagtgctcggtgccttgaag

SEQ ID NO: 74 - DNA, 23 bp, synthetic construct
  source 1..23: mol_type = other DNA
cccttggacccagaggttctgtg

SEQ ID NO: 75 - DNA, 25 bp, synthetic construct
  source 1..25: mol_type = other DNA
ccccttggacccagaggttcttata

SEQ ID NO: 76 - DNA, 28 bp, synthetic construct
  source 1..28: mol_type = other DNA
tctacccttggacccagaggttcttgga

SEQ ID NO: 77 - DNA, 23 bp, synthetic construct
  source 1..23: mol_type = other DNA
tctacccttggacccagagggtg

SEQ ID NO: 78 - DNA, 25 bp, synthetic construct
  source 1..25: mol_type = other DNA
ttggtctattttcccacccttatgc

SEQ ID NO: 79 - DNA, 26 bp, synthetic construct
  source 1..26: mol_type = other DNA
ttggtctattttcccacccttagttg

SEQ ID NO: 80 - DNA, 23 bp, synthetic construct
  source 1..23: mol_type = other DNA
ttgcactggtggggtgaattctt

SEQ ID NO: 81 - DNA, 24 bp, synthetic construct
  source 1..24: mol_type = other DNA
cagtgataatttctgggttaatgc

SEQ ID NO: 82 - DNA, 29 bp, synthetic construct
  source 1..29: mol_type = other DNA
ataacagtgataatttctgggttaagtta

SEQ ID NO: 83 - DNA, 20 bp, synthetic construct
  source 1..20: mol_type = other DNA
cttctgcatcctgtcggcaa

SEQ ID NO: 84 - DNA, 20 bp, synthetic construct
  source 1..20: mol_type = other DNA
gactgtctcccggctctgcc

SEQ ID NO: 85 - DNA, 25 bp, synthetic construct
  source 1..25: mol_type = other DNA
taggaagtcaccaaagcagtacagc

SEQ ID NO: 86 - DNA, 23 bp, synthetic construct
  source 1..23: mol_type = other DNA
tacttgtaccagctcactaccta

SEQ ID NO: 87 - DNA, 19 bp, synthetic construct
  source 1..19: mol_type = other DNA
atgacacctgcttgctctc

SEQ ID NO: 88 - DNA, 25 bp, synthetic construct
  source 1..25: mol_type = other DNA
aatccatcctatctgggaacattag

SEQ ID NO: 89 - DNA, 20 bp, synthetic construct
  source 1..20: mol_type = other DNA
cctttatagcaataaattca

SEQ ID NO: 90 - DNA, 20 bp, synthetic construct
  source 1..20: mol_type = other DNA
atgtaacgaaaatctagggc

SEQ ID NO: 91 - DNA, 24 bp, synthetic construct
  source 1..24: mol_type = other DNA
ccctgggctctgtaaagaatagtg

SEQ ID NO: 92 - DNA, 24 bp, synthetic construct
  source 1..24: mol_type = other DNA
atcagagcttaaactgggaagctg

SEQ ID NO: 93 - DNA, 21 bp, synthetic construct
  source 1..21: mol_type = other DNA
gaatattcccgctctccggag

SEQ ID NO: 94 - DNA, 22 bp, synthetic construct
  source 1..22: mol_type = other DNA
gctggtgctccattcttgagtg

(19) United States
(12) Patent Application Publication
(10) Pub. No.: US 2026/0185160 A1
(43) Pub. Date: Jul. 2, 2026

(54) KITS AND METHODS FOR DETERMINING GENO TYPE OF THALASSEMIA

(71) Applicant: Origin Biotechnology Co., Ltd., Taipei City (TW)

(72) Inventors: Zing-Wei LOONG, Taipei City (TW); Yu-Wei CHEN, Taipei City (TW); I-Fan CHIU, Taipei City (TW)

(21) Appl. No.: 19/430,382

(22) Filed: Dec. 23, 2025

(30) Foreign Application Priority Data
Dec. 31, 2024 (CN) 202411981565.2

(51) Int. Cl. C12Q 1/6883; C12Q 1/48; C12Q 1/6851; C12Q 1/686

(52) U.S. Cl. CPC C12Q 1/6883; C12Q 1/48; C12Q 1/6851; C12Q 1/686; C12Y 207/07; C12Q 2600/112; C12Q 2600/156; C12Q 2600/16

(57) ABSTRACT

Provided herein are kits and methods for determining a thalassemia genotype of a subject. The present kit includes at least primers that target ζ2, Ψζ1, Ψα2, Ψα1, α2, α1 and θ1 genes in α-globin gene cluster, and primers that target variations in α2-globin gene and β-globin gene. Also encompasses herein is a method for determining a thalassemia genotype of a subject, in which gene copy number of each target sites in ζ2, Ψζ1, Ψα2, Ψα1, α2, α1 and θ1 genes in α-globin gene cluster are determined by use of the present kit, and compared with those of normal or known thalassemia subject to arrive at the thalassemia genotype of the subject.

### REFERENCE TO A SEQUENCE LISTING

The present application is being filed along with a Sequence Listing in electronic format. The Sequence Listing is provided as a file entitled “P4463_SeqList_AF”, created on Dec. 23, 2025, which is 83,911 bytes in size. The information in the electronic format of the Sequence Listing is incorporated herein by reference in its entirety.

### CROSS-REFERENCES TO RELATED APPLICATIONS

This application claims priority and the benefit of China Patent Application No. 202411981565.2, filed Dec. 31, 2024, the entirety of which is incorporated herein by reference

### BACKGROUND OF THE INVENTION

### 1. Field of the Invention

The present disclosure relates to the field of disease diagnosis. More particularly, the present disclosure relates to a kit comprising primers with specific polynucleotide sequences, and uses of the kit in the identification of genotypes of thalassemia subjects.

### 2. Description of Related Art

Thalassemia is a blood disorder that is inherited. When one has thalassemia, his/her body makes less hemoglobin than normal. Hemoglobin is an iron-rich protein in red blood cells. It carries oxygen to all parts of the body. Normal adult hemoglobin comprises four globin proteins, two of which are alpha (α) proteins and two of which are beta (β) proteins. There are 2 main types of thalassemia: α- and β-thalassemia respectively caused by gene mutations in α- and β-globins. Thalassemia is common in multiple geographic locations including Greece, Cyprus, sub-Saharan countries, Arabic countries, India, and Southeast Asia.

Normal individuals have 2α genes on each chromosome 16 (αα/αα) and they are located on the short arm. Alpha-thalassemia is due to either deletional or non-deletional mutation on at least one of the four α-globin genes. Common deletional types include single-gene losses (−α^{3.7}, −α^{4.2}) and double-gene losses like Southeast Asian (−/^{SEA}), Mediterranean (−/^{MED} or α⁰−), and Filipino (−/^{FIL}) deletions. On the other hand, non-deletional forms are caused by point mutations on the «2 globin gene or α¹ globin gene, such as α^{WS}α (α2:c.369C>G), α^{QS}α (α2:c.377T>C), α^{CS}α (α2:c.427T>C). There are 4 types of α-thalassemia, ranging from trait (deletion of one or two α-globin genes) to α-thalassemia major (all four α-globin genes are deleted), resulting in severe transfusion-dependent anemia. Two clinically significant forms of α-thalassemia are hemoglobin Bart hydrops fetalis (Hb Bart) syndrome (caused by deletion/inactivation of all four α-globin alleles; −/−), and hemoglobin H (HbH) disease (most frequently caused by deletion/inactivation of three α-globin alleles; −/−α).

Beta-thalassemia is caused by either deletional or non-deletional mutations in the β-globin gene located on chromosome 11. The mutations can be nucleotide substitutions (e.g., β:c.52A>T, β:c.79G>A, β:c.92+1G>T, β: c.−78A>G, β: c.−79A>G), frameshift insertions/deletions (e.g., β:c.124_127delTTCT, β:c.216-217insA) or gross deletions within the β-globin gene, leading to reduced or absent β-globin chain synthesis, which in turn causes imbalance in the α/β globin chain ratio. Depending on the severity of the mutation, β-thalassemia also can be classified as β-thalassemia minor (trait), β-thalassemia intermedia, or β-thalassemia major.

Carriers of thalassemia genes may have no symptoms (thalassemia minor) or very mild symptoms with occasional crisis (thalassemia intermedia) that require blood transfusion. As to individuals who are homozygous for the mutation have severe and life threatening symptoms (thalassemia major). Fetus having homozygous mutations are often stillbirth during the pregnancy (23 to 38 weeks) or dead within half hour after birth, and mothers of such fetus often have pre-eclampsia, premature birth or abnormal breeding. Accordingly, knowing the genotype of each thalassemia individual is an important step in prevention as well as treatment strategies.

In view of the above, there exists in the related art a need of an improved method and/or kit for the identification of the genotype of an individual having or suspected of having thalassemia, so that customary therapeutic strategy may be designed and administered to a thalassemia subject or a genetic counseling with risk assessment may be provided to a thalassemia carrier to reduce or prevent maternal transmission.

### SUMMARY

The present disclosure aims to provide primers and methods for determining thalassemia genotype of a subject. Thus, the first aspect of the present disclosure is directed to a kit for determining a genotype of a subject having thalassemia. The kit comprises:
a first group of primers respectively having the nucleic acid sequences of SEQ ID Nos: 1-38 for targeting 19 target sites ζ2, ψζ, Ψα2, Ψα1, α2, α1 and θ1 genes in α-globin gene cluster;
a second group of primers respectively having the nucleic acid sequences of SEQ ID Nos: 39-44 for targeting α^{WS}α, α^{QS}α, and α^{CS}α variants in the α2-globin gene;
a third group of primers respectively having the nucleic acid sequences of SEQ ID Nos: 45-82 for targeting β⁻³², β⁻³⁰, β⁻²⁹, β⁻²⁸, β^{Cap+1}, β^{IntM}, β^{CD14-15}, β^{CD17}, β^{CD26}, β^{CD27/28}, β^{IVS-I-1(G>T)}, β^{IVS-I-1(G>A)}, β^{IVS-1-5}, β^{CD71-72}, β^{CD43}, β^{CD41-42}, β^{CD31}, and β^{IVS-II-654} variants in β-globin gene;
a fourth group of primers respectively having the nucleic acid sequence of SEQ ID Nos: 83-90 for targeting a reference gene; and
a fifth group of primers respectively having the nucleic acid sequence of SEQ ID Nos: 91-94 for targeting a chromosomal gene.

Examples of the reference gene suitable for use in the present kit include, but are not limited to, glyceraldehyde-3-phosphate dehydrogenase (GAPDH), actin beta (ACTB), cystic fibrosis transmembrane conductance regulator (CFTR), hypoxanthine-guanine phosphoribosyltransferase (HPRT), ribonuclease P protein subunit p30 (RPP30), ribonuclease P protein subunit p40 (RPP40), and a combination thereof.

Examples of the chromosomal gene suitable for use in the present kit include, but are not limited to, amelogenin (AMEL), zinc finger protein, X-linked (ZFX), zinc finger protein, Y-linked (ZFY), TATA-box binding protein associated factor 9 (TAF9), sex-determining region Y protein (SRY), and a combination thereof.

According to embodiments of the present disclosure, at least one primer is labeled with a fluorescent molecule. Examples of the fluorescent molecule suitable for use in the present kit include, but are not limited to carboxyfluorescein (FAM), 2′-chloro-7′-phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX). In some examples, at least one primer is labeled with FAM. In other examples, at least one primer is labeled with NED. In further examples, 24 primers are independently labeled with FAM, and 3 primers are independently labeled with NED.

According to optional embodiments of the present disclosure, the kit may further include an amplification reagent, a normal control, a positive control, and a blank control.

According to embodiments of the present disclosure, the amplification reagent may be one or more agents selected from the group consisting of a buffer, a hot start DNA polymerase, deoxyadenosine triphosphate (dATP), deoxycytidine triphosphate (dCTP), deoxyguanosine triphosphate (dGTP), deoxythymidine triphosphate (dTTP), and betaine.

According to embodiments of the present disclosure, the normal control is a genomic DNA of a healthy subject, the positive control is a genomic DNA of a subject of α-thalassemia or β-thalassemia; and the blank control is a buffer solution (e.g., TRIS buffer of pH 8.5).

The second aspect of the present disclosure aims to a method for determining a genotype of a subject having thalassemia via use of the present kit. The method includes steps of:
(a) mixing a nucleic acid sample with a primer mixture and an amplification reagent to produce a reaction mixture, wherein the primer mixture consists of the first, second, third, fourth and fifth groups of primers of the present kit; and the nucleic acid sample is a genomic DNA isolated from the subject, a healthy subject or an α-thalassemia or β-thalassemia subject;
(b) subjecting the reaction mixture of step (a) to a polymerase chain reaction (PCR) to produce amplicons;
(c) subjecting the amplicons to capillary electrophoresis to separate the amplicons from one another thereby generating a plurality of peaks independently corresponds to one separated amplicon;
(d) determining the peak area of each separated amplicon of step (c), in which each separated amplicon corresponds to a gene targeted by the first, second, third, fourth, or fifth groups of primers;
(e) calculating a peak ratio (R) of the gene targeted by the first, second, or third groups of primers in step (d);
(f) determining a copy number of the gene targeted by the first, second or third groups of primers based on the calculated R of step (e); and
(g) determining the genotype of the subject based on the determined copy number in step (f);
wherein,
in step (e), the R is calculated by,
(i) dividing the peak area of each separated amplicon corresponds to the gene targeted by the first, second or third groups of primers with the sum of the peak area of an internal control to generate a first value; and
(ii) dividing the first value of step (i) with a second value derived from the same gene in the normal control; and
in step (f), the copy number of the gene is 0 when R is ≤0.35, the copy number of the gene is 1 when 0.35<R≤1.42, the copy number of the gene is 2 when 1.42<R≤2.68 or the copy number of the gene is 3 when R>2.68.

According to embodiments of the present disclosure, the primer is labeled with a fluorescent molecule. Examples of the fluorescent molecules suitable for use in the present kit include, but are not limited to FAM, VIC, HEX, JOE, TMR, NED, and ROX. In certain examples, at least one primer is labeled with FAM. In other examples, at least one primer is labeled with NED.

According to embodiments of the present disclosure, the amplification reagent may be one or more agents selected from the group consisting of a buffer, a hot start DNA polymerase, dATP, dCTP, dGTP, dTTP, and betaine.

According to embodiments of the present disclosure, in step (b), the PCR is performed under the conditions of: (1) 95° C. for 5 minutes; (2) 28 cycles of the followings: 95° C. for 30 seconds, 66° C. for 40 seconds, and 72° C. for 40 seconds; (3) 72° C. for 45 minutes; and (4) 4° C.

Optionally or in addition, the method further comprises repeating steps (a) to (d) by use of a buffer solution (e.g., TRIS buffer of pH 8.5), which serves as a blank control of the present method.

According to embodiments of the present disclosure, in step (e) (i), the internal control is one or more reference genes independently selected from the group consisting of glyceraldehyde-3-phosphate dehydrogenase (GAPDH), actin beta (ACTB), cystic fibrosis transmembrane conductance regulator (CFTR), hypoxanthine-guanine phosphoribosyltransferase (HPRT), ribonuclease P protein subunit p30 (RPP30), ribonuclease P protein subunit p40 (RPP40), and a combination thereof. According to preferred embodiments of the present disclosure, the internal control consists of ACTB, CFTR, RPP30 and RPP40 genes.

According to embodiments of the present disclosure, in step (g),
the subject has heterozygous deletion form of thalassemia with −α^{3.7}/αα genotype when the gene copy number is 1 for each target sites Nos. 10-12, and the gene copy number is 2 for each of the rest target site;
the subject has heterozygous deletion form of thalassemia with −α^{4.2}/αα genotype when the gene copy number is 1 for each target site Nos. 9-10, and the gene copy number is 2 for each of the rest target sites;
the subject has heterozygous deletion form of thalassemia with −^{SEA}/αα genotype when the gene copy number is 1 for each target site Nos.: 8-18, and the gene copy number is 2 for each of the rest target sites;
the subject has heterozygous deletion form of thalassemia with −^{THA1}/αα genotype when the gene copy number is 1 for each target site Nos: 4-17 and the gene copy number is 2 for each of the rest target sites;
the subject is determined to have heterozygous deletion form of thalassemia with −^{FIL}/αα genotype when the gene copy number is 1 for each target site Nos: 5-16, and the gene copy number is 2 for each of the rest target sites;
the subject has heterozygous deletion form of thalassemia with −^{MED-I}/αα genotype when the gene copy number is 1 for each target site Nos: 7-15, and the gene copy number is 2 for each of the rest target sites;
the subject has heterozygous deletion form of thalassemia with −^{MED-II}/αα genotype when the gene copy number is 1 for each target site Nos: 3-14, and the gene copy number is 2 for each of the rest target sites;
the subject has heterozygous deletion form of thalassemia with −α^{20.5}/αα genotype when the gene copy number is 1 for each target site Nos: 6-12 and the gene copy number is 2 for each of the rest target sites;
the subject has homozygous deletion form of thalassemia with −α^{3.7}/−α^{3.7} genotype when the gene copy number is 0 for each target site Nos: 10-12, and the gene copy number is 2 for each of the rest target sites;
the subject has homozygous deletion form of thalassemia with −α^{4.2}/−α^{4.2} genotype when the gene copy number is 0 for each target site Nos: 9-10, and the gene copy number is 2 for each of the rest target sites;
the subject has homozygous deletion form of thalassemia with −^{SEA}/−^{SEA} genotype when the gene copy number is 0 for each target site Nos: 7-18, and the gene copy number is 2 for each of the rest target sites;
the subject has homozygous deletion form of thalassemia with −^{THA1}/−^{THA1} genotype when the gene copy number is 0 for each target site Nos: 4-17, and the gene copy number is 2 for each of the rest target sites;
the subject has homozygous deletion form of thalassemia with −^{FIL}/−^{FIL} genotype when the gene copy number is 0 for each target site Nos: 5-16, and the gene copy number is 2 for each of the rest target sites;
the subject has homozygous deletion form of thalassemia with −^{MED-I}/−^{MED-I} genotype when the gene copy number is 0 for each target site Nos: 7-15, and the gene copy number is 2 for each of the rest target sites;
the subject has homozygous deletion form of thalassemia with −^{MED-II}/−^{MED-II} genotype when the gene copy number is 0 for each target site Nos: 3-14, and the gene copy number is 2 for each of the rest target sites;
the subject has homozygous deletion form of thalassemia with −α^{20.5}/−α^{20.5} genotype when the gene copy number is 0 for each target site Nos: 6-12, and the gene copy number is 2 for each of the rest of target sites;
the subject has duplication form of thalassemia with ααα ^{anti-3.7}/αα genotype when the gene copy number is 3 for each target site Nos: 10-12, and the gene copy number is 2 for each of the rest target sites;
the subject has duplication form of thalassemia with ααα ^{anti-4.2}/αα genotype when the gene copy number is 3 for each target site Nos: 9-10, and the gene copy number is 2 for each of the rest target sites;
the subject has HS-40 heterozygous deletion form of thalassemia with del HS-40 αα/αα genotype when the gene copy number is 1 for the target site No: 1, and the gene copy number is 2 for each the rest target sites;
the subject has HS-40 homozygous deletion form of thalassemia with del HS-40/del HS-40 αα/αα genotype when the gene copy number is 0 for the target site No: 1, and the gene copy number is 2 for each of the rest target sites;
the subject has homozygous deletion of β-globin gene when the gene copy number for β-globin is 0; or
the subject has heterozygous deletion of β-globin gene when the gene copy number of β-globin is 1.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a schematic diagram depicting the present primer targeting sites in α- and β-globin gene clusters in accordance with one embodiment of the present disclosure;

FIG. 2 depicts the identification results of a normal male subject (αα/αα genotype) in accordance with one embodiment of the present disclosure;

FIG. 3 depicts the identification results of −α^{3.7}/αα deletional form of α-thalassemia male subject in accordance with one embodiment of the present disclosure;

FIG. 4 depicts the identification results of −^{SEA}/αα deletional form of α-thalassemia female subject in accordance with one embodiment of the present disclosure;

FIG. 5 depicts the identification results of α^{WS}α/αα non-deletional form of α-thalassemia female subject in accordance with one embodiment of the present disclosure; and

FIG. 6 depicts the identification results of β^{CD41-42} (c. 126_129delCTTT)/β^{N} non-deletional form of β-thalassemia female subject in accordance with one embodiment of the present disclosure.

In accordance with common practice, the various described features/elements are not drawn to scale but instead are drawn to best illustrate specific features/elements relevant to the present invention.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

Ranges of values are disclosed herein. The ranges set out a lower limit value and an upper limit value. Unless otherwise stated, the ranges include all values to the magnitude of the smallest values (either lower limit value or upper limit value) and ranges between the values of the stated ranges.

The singular forms “a”, “and”, and “the” are used herein to include plural referents unless the context clearly dictates otherwise.

As used herein, the term “polynucleotide sequence” is understood to mean either a double-stranded DNA or a single-stranded DNA. The polynucleotide sequences of the invention can be isolated, purified (or partially purified), by separation methods including, but not limited to, ion-exchange chromatography, molecular size exclusion chromatography, or by genetic engineering methods such as amplification, subtractive hybridization, cloning, sub-cloning or chemical synthesis, or combinations of these genetic engineering methods.

As used herein, the term “amplification reagents” refers to the chemicals, apart from the specified primers (i.e., the first to the fifth primers of the present kit), needed to perform the PCR process. These chemicals generally comprise four classes of components: (i) an aqueous buffer (also known as PCR buffer), (ii) a water soluble magnesium salt (e.g., MgCl₂), (iii) at least four deoxyribonucleotide triphosphates (dNTPs, including thymidine triphosphate (dTTP), deoxyadenosine triphosphate (dATP), deoxycitidine triphosphate (dCTP) and deoxyguanosine triphosphate (dGTP)), and (iv) a polynucleotide polymerase, preferably a DNA polymerase, more preferably a thermostable DNA polymerase, i.e., a DNA polymerase, which can tolerate temperatures between 90° C. and 100° C. for a total time of at least 10 minutes without losing more than about half its activity. Depending on desired purposes, these chemicals may comprise additional components for improving the efficacy and/or specificity of the PCR process, such as betaine, ethylene glycol, and glycerol.

The term “subject” refers to a human species diagnosed by the kit and/or method of the present invention. The term “subject” is intended to refer to both the male and female gender unless one gender is specifically indicated.

### II. Description of the Invention

The present disclosure aims at providing kits and methods for accurately and efficiently determining genotype of a thalassemia subject, so that customary therapeutic strategy may be designed and administered to the subject to ameliorate symptoms associated with thalassemia, or a genetic counseling with risk assessment may be provided to a thalassemia carrier to reduce or prevent maternal transmission.

As indicated above, thalassemia is caused by mutations in α- or β-globin genes. Thus, inventors of the present disclosure design and synthesize primers respectively targeting α-globin gene cluster and β-globin gene to obtain copy numbers of target genes, which are then used to determine the thalassemia genotype of a subject.

### (i) The Present Kit

Accordingly, the first aspect of the present disclosure aims to provide a kit, which comprises a first, a second and a third groups of primers respectively targeting α-globin gene cluster, α2-globin gene and β-globin gene.

According to embodiments of the present disclosure, the first group of primers has a total of 38 primers respectively having the nucleic acid sequences of SEQ ID Nos: 1-38, which are designed to amplify nucleic acid sequences in 19 target sites respectively disposed in the upstream and downstream of ζ2, Ψζ1, Ψα2, Ψα1, α2, α1 and θ1 genes in the α-globin gene cluster, accordingly, DNA fragments or amplicons of the 19 target sites are produced after amplification by polymerase chain reaction (PCR). The second group of primers has a total of 6 primers respectively having the nucleic acid sequences of SEQ ID Nos: 39-44, which are designed to amplify nucleic acid sequences having α^{WS}α, α^{QS}α, or α^{CS}α variants in the α2-globin gene, accordingly, amplicons having α^{WS}α, α^{QS}α, or α^{CS}α variants are produced after PCR amplification. The third group of primers has a total of 38 primers respectively having the nucleic acid sequences of SEQ ID Nos: 45-82, which are designed to amplify nucleic acid sequences comprising β⁻³², β⁻³⁰, β⁻²⁹, β⁻²⁸, β^{Cap+1}, β^{IntM},β^{CD14-15}, β^{CD17}, β^{CD26}, β^{CD27/28}, β^{IVS-I-1(G>T)}, β^{IVS-I-1(G>A)}, β^{IVS-I-5}, β^{CD71-72}, β^{CD43}, β^{CD41-42}, β^{CD31}, or β^{IVS-II-654} variants in β-globin gene, accordingly, amplicons comprising any one of the indicated variants of β-globin gene are produced after PCR amplification.

Additionally, the kit may further comprise a fourth and a fifth groups of primers respectively targeting a reference gene and a chromosomal gene. According to embodiments of the present disclosure, the fourth group of primers has a total of 8 primers respectively having the nucleic acid sequence of SEQ ID Nos: 83-90, which are designed to amplify nucleic acid sequences of a reference gene. Accordingly, amplicons of the reference gene are produced after PCR amplification. Examples of the reference gene suitable for use in the present kit include, but are not limited to, glyceraldehyde-3-phosphate dehydrogenase (GAPDH), actin beta (ACTB), cystic fibrosis transmembrane conductance regulator (CFTR), hypoxanthine-guanine phosphoribosyltransferase (HPRT), ribonuclease P protein subunit p30 (RPP30), ribonuclease P protein subunit p40 (RPP40), and a combination thereof. The fifth group of primers has a total of 4 primers respectively having the nucleic acid sequence of SEQ ID Nos: 91-94, which are designed to amplify nucleic acid sequences of a chromosomal gene. Accordingly, amplicons of the chromosomal gene are produced after PCR amplification. Examples of the chromosomal gene suitable for use in the present kit include, but are not limited to, amelogenin (AMEL), zinc finger protein, X-linked (ZFX), zinc finger protein, Y-linked (ZFY), TATA-box binding protein associated factor 9 (TAF9), sex-determining region Y protein (SRY), and a combination thereof.

According to further embodiments of the present disclosure, at least one primer is labeled with a fluorescent molecule. Examples of the fluorescent molecule suitable for use in the present kit include, but are not limited to carboxyfluorescein (FAM), 2′-chloro-7′-phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX). In some embodiments, at least one primer is labeled with FAM. In other embodiments, at least one primer is labeled with NED. In certain embodiments, 24 primers are independently labeled with FAM, while 3 primers are independently labeled with NED.

According to optional embodiments of the present disclosure, the kit may further include an amplification reagent, a normal control, a positive control, and a blank control. According to embodiments of the present disclosure, the amplification reagent may be one or more agents selected from the group consisting of a buffer solution, a hot start DNA polymerase, dATP, dCTP, dGTP, dTTP, and betaine. According to embodiments of the present disclosure, the normal control is a genomic DNA of a healthy subject, the positive control is a genomic DNA of a subject of α-thalassemia or β-thalassemia; and the blank control is a buffer solution (e.g., TRIS buffer of pH 8.5).

### (ii) the Present Method

The present disclosure also encompasses a method for determining a genotype of a subject having or suspected of having thalassemia via use of the present primer kit in section (i) of this paper. To this purpose, the gene copy numbers of various target genes in α-globin gene cluster and β-globin gene of a candidate subject are determined and compared with those of normal or known thalassemia subjects thereby generating an identification result (i.e., the thalassemia genotype) for the candidate subject.

The present method commences by mixing a nucleic acid sample with a primer mixture and an amplification reagent to produce a reaction mixture, wherein the primer mixture consists of the first, second, third, fourth and fifth groups of primers of the present kit; and the nucleic acid sample is a genomic DNA isolated from the subject, a healthy subject or an α-thalassemia or β-thalassemia subject (step (a)).

According to embodiments of the present disclosure, the nucleic acid sample of the subject is extracted from a cell or tissue of the human subject. The cell or tissue may be any available cell or tissue obtained from the subject, as long as such the cell or tissue contains the DNA of the subject. For example, the cell may be an epithelial cell, fibroblast, stem cell, blood cell, keratinocyte, or adipocyte. The tissue may be a tissue biopsy, such as a gastric, esophageal, colorectal, brain, hepatic, splenic, or skin biopsy. According to some embodiments of the present disclosure, the nucleic acid sample may be extracted from the blood sample of the subject. The nucleic acid sample may be extracted from the cell or tissue by a commercial kit, or any conventional DNA extraction technique; for example, the phenol/chloroform assay, and detergent (e.g., sodiumdodecyl sulfate, Tween-20, NP-40, and Triton X-100)/acetic acid assay. Genomic DNA from a healthy subject or a thalassemia subject respectively serving as a normal control and a positive control in the present method may be extracted by the same manner.

Then, the extracted nucleic acid sample is then mixed with a primer mixture and an amplification reagent to produce a reaction mixture. Specifically, the primer mixture consists of the first, second, third, fourth and fifth groups of primers of the present kit described in section (i) of this paper, while the normal control is a genomic DNA of a healthy subject, and the positive control is a genomic DNA of a subject of α-thalassemia or β-thalassemia. According to embodiments of the present disclosure, the amplification reagent is a combination of a PCR buffer solution (e.g., Taq buffer), a hot start DNA polymerase (e.g., Taq polymerase), dATP, dCTP, dGTP, dTTP, MgCl₂, and betaine. The reaction mixture is then subjected to PCR to produce amplicons (step (b)).

According to embodiments of the present disclosure, the PCR is performed under the conditions of: (1) 95° C. for 5 minutes; (2) 28 cycles of the following: 95° C. for 30 seconds, 66° C. for 40 seconds, and 72° C. for 40 seconds; (3) 72° C. for 45 minutes; and (4) 4° C. According to embodiments of the present disclosure, after step (b), the following amplicons are produced, including amplicons of 19 target sites respectively disposed in the upstream and downstream of ζ2, Ψζ1, Ψα2, Ψα1, α2, α1 and θ1 genes in the α-globin gene cluster; amplicons having α^{WS}α, α^{QS}α, or α^{CS}α variants; amplicons comprising β⁻³², β⁻³⁰, β⁻²⁹, β⁻²⁸, B^{Cap+1}, B^{IntM}, β^{CD14-15}, β^{CD17}, β^{CD26}, β^{CD27/28}, β^{IVS-1(G>T)}, β^{IVS-1(G>A)}, β^{IVS-I- 5}, β^{CD71-72}, β^{CD43}, β^{CD41-42}, β^{CD31}, or β^{IVS-II-654} variants; amplicons of the genomic DNA of a healthy subject (i.e., normal control); and amplicons of the genomic DNA of α-thalassemia or β-thalassemia subject (i.e., positive control) are produced.

The thus produced amplicons in step (b) are then subjected to capillary electrophoresis to separate one amplicon from the other thereby generates a plurality of peaks independently corresponds to one separated amplicon (step (c)). Then, the peak area of each peak correspond to one separated amplicon is determined (step (d)).

Optionally or in addition, the afore described steps (a) to (d) are repeated by use of a buffer solution, such as TRIS buffer of pH 8.5, which serves as a blank control in the present method.

To obtain the copy number of each target gene (e.g., genes targeted by the first, second, or third groups of primers), a peak ratio (R) of each target gene is calculated (step (e)). According to embodiments of the present disclosure, the R is calculated by following steps:
(i) dividing the peak area of each separated amplicon corresponds to the gene targeted by the first, second or third groups of primers with the sum of the peak area of an internal control to generate a first value; and
(ii) dividing the first value of step (i) with a second value derived from the same gene in the normal control.

According to embodiments of the present disclosure, in step (i), the internal control is one or more reference genes independently selected from the group consisting of glyceraldehyde-3-phosphate dehydrogenase (GAPDH), actin beta (ACTB), cystic fibrosis transmembrane conductance regulator (CFTR), hypoxanthine-guanine phosphoribosyltransferase (HPRT), ribonuclease P protein subunit p30 (RPP30), ribonuclease P protein subunit p40 (RPP40), and a combination thereof. In certain embodiments, the internal control consists of 4 reference genes, which are respectively ACTB, CFTR, RPP30 and RPP40.

The calculated R of each target gene is then used to determine the gene copy number of each target gene, in which the target gene has a copy number of 0, when R is equal to or less than 0.35 (R≤0.35), or the target gene has a copy number of 1, when R is greater than 0.35 and equal to or smaller than 1.42 (0.35<R≤1.42), or the target gene has a copy number of 2, when R is greater than 1.42 and equal to or smaller than 2.68 (1.42<R≤2.68), or the target gene has a copy number of 3, when R is greater than 2.68 (R>2.68) (step (f)).

The thus obtained gene copy number of each target gene of the subject is then compared with that of a normal or known thalassemia subject thereby determining the genotype of the subject (step (g)).

According to embodiments of the present disclosure, when the 19 target sites (i.e., nucleic acids recognized and amplified by the first group of primers of the present kit) respectively have 2 copy of genes, then the subject is normal without thalassemia; or when target sites #10-12 independently have 1 copy of gene, while the rest of the target sites respectively have 2 copy of genes, then the subject has heterozygous deletion form of thalassemia with −α^{3.7}/αα genotype; or when target sites #9 and 10 respectively have 1 copy of gene, while the rest of the target sites respectively have 2 copy of genes, then the subject has heterozygous deletion form of thalassemia with −α^{4.2}/αα genotype; or when target sites #8-18 independently have 1 copy of gene, while the rest of the target sites respectively have 2 copy of genes, then the subject has heterozygous deletion form of thalassemia with −^{SEA}/αα genotype; or when target sites #4-17 independently have 1 copy of gene, while the rest of the target sites respectively have 2 copies of genes, then the subject has heterozygous deletion form of thalassemia with −^{THA1}/αα genotype; or when target sites #5-16 independently have 1 copy of gene, while the rest of the target sites respectively have 2 copy of genes, then the subject is determined to have heterozygous deletion form of thalassemia with −^{FIL}/αα genotype; or when target sites #7-15 independently have 1 copy of gene, while the rest of the target sites respectively have 2 copy of genes, then the subject has heterozygous deletion form of thalassemia with −^{MED-I}/αα genotype; or when target sites #3-14 independently have 1 copy of gene, while the rest of the target sites respectively have 2 copy of genes, then the subject has heterozygous deletion form of thalassemia with −^{MED-I}/αα genotype; or when target sites #6-12 independently have 1 copy of gene, while the rest of the target sites respectively have 2 copy of genes, then the subject has heterozygous deletion form of thalassemia with −α^{20.5}/αα genotype. Similarly, when target sites #10 to 12 respectively have 0 copy of gene, while the rest of the target sites independently has 2 copy of genes, then the subject has homozygous deletion form of thalassemia with −α^{3.7}/−α^{3.7} genotype; or when target sites #9-10 respectively have 0 copy of gene, while the rest of the target sites independently has 2 copy of genes, then the subject has homozygous deletion form of thalassemia with −α^{4.2}/−α^{4.2} genotype; or when target sites #7 to 18 respectively have 0 copy of gene, while the rest of the target sites independently has 2 copy of genes, then the subject has homozygous deletion form of thalassemia with −^{SEA}/−^{SEA} genotype; when target sites #4 to 17 respectively have 0 copy of gene, while the rest of the target sites independently has 2 copy of genes, then the subject has homozygous deletion form of thalassemia with −^{THA1}/−^{THA1} genotype; or when target sites #5 to 16 respectively have 0 copy of gene, while the rest of the target sites independently has 2 copy of genes, then the subject has homozygous deletion form of thalassemia with −^{FIL}/−^{FIL} genotype; or when target sites #7 to 15 respectively have 0 copy of gene, while the rest of the target sites independently has 2 copy of genes, then the subject has homozygous deletion form of thalassemia with −^{MED-I}/−^{MED-I} genotype; or when target sites #3 to 14 respectively have 0 copy of gene, while the rest of the target sites independently has 2 copy of genes, then the subject has homozygous deletion form of thalassemia with −^{MED-II}/−^{MED-II} genotype; or when target sites #6 to 12 respectively have 0 copy of gene, while the rest of the target sites independently has 2 copy of genes, then the subject has homozygous deletion form of thalassemia with −α^{20.5}/−α^{20.5} genotype. Alternatively, when target sites #10 to 12 respectively have 3 copy of genes, while the rest of the target sites independently has 2 copy of genes, then the subject has duplication form of thalassemia with ααα ^{anti-3.7}/αα genotype; or when target sites #9 and 10 respectively have 3 copy of genes, while the rest of the target sites independently has 2 copy of genes, then the subject has duplication form of thalassemia with ααα ^{anti-4.2}/αα genotype. Still more alternatively, when target site #1 has 1 copy of genes, while the rest of the target sites independently has 2 copy of genes, then the subject has HS-40 heterozygous deletion form of thalassemia with del HS-40 αα/αα genotype; or when target site #1 has 0 copy of genes, while the rest of the target sites independently has 2 copy of genes, then the subject has HS-40 homozygous deletion form of thalassemia with del HS-40 αα/αα genotype.

According to embodiments of the present disclosure, when the β-globin (i.e., nucleic acids recognized and amplified by the third group of primers of the present kit) has the copy number of 0, then the subject has homozygous deletion of β-globin gene; when the β-globin has the copy number of 1, the subject has heterozygous deletion of β-globin gene.

Accordingly, by amplifying target sites in α-globin gene cluster and/or β-globin gene of a candidate subject with the primers of the present kit to obtain the gene copy number in each target sites, then compare the thus obtained gene copy numbers of each and every target sites with those of the normal or known thalassemia subject, a skilled artisan may easily identify a normal subject, a thalassemia subject, and/or differentiate a thalassemia subject from a thalassemia carrier.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Example 1: Preparation of the Present Primers

Based on α- and β-globin gene clusters provided in NCBI public database, primers directed to the upstream and downstream of ζ2, Ψζ1, Ψα2, Ψα1, α2, α1 and θ1 genes of the α-globin gene cluster, β-globin gene and four internal controls were designed and synthesized. The nucleotide sequences of the synthesized primers are listed in Table 1. The reaction buffer and primer mixtures were prepared in accordance with the conditions provided in Tables 2 and 3, the thus prepared reaction buffer and primer mixtures were stored at −20±5° C. until further use.

TAßLE 1
Sequences of the present primers and their target sites

| SEQ ID NO | Sequence (5′-3′) | fluorophore | Target site |
|---|---|---|---|
| 1 | CACATCTGCCCAAGCCAAGG | carboxyfluorescein | HS-40 |
| | | (FAM) | |
| 2 | CTGTTGGCCTCCAGAAGCAC | | |
| 3 | CTGTTCCCCCGGTTACCCTC | FAM | Downstream of |
| 4 | GTCCCTGGGCCTTGGTTTG | | HS-40 |
| 5 | CTGCATCATAATTCCAGCAGGA | FAM | Upstream of ζ2 |
| 6 | CTCTCGCTTTATTCTTCCTTTTC | | gene |
| 7 | GGATAACTTCCCTATCAGATA | FAM | |
| 8 | GTGTCTATCCTTTACCACAG | | |
| 9 | AGAACTGGACTACAAATGCAGGAGT | FAM | ζ2 gene |
| 10 | GGGGCTATCACTCCTGTTCCTC | | |
| 11 | AGGCTGGGCATGGTGGCTCATA | FAM | Upstream of Ψζ1 |
| 12 | CGCGCCCGGCCTTAATTTTGT | | gene |
| 13 | GGACAGTGGAGACAGATAGTCT | FAM | Ψζ1 gene |
| 14 | CCTTCAGGCCTAGAACGAAAC | | |
| 15 | TCAGTGCCAGTGACCTGTTG | FAM | Ψα2 gene |
| 16 | TGTCAGAGCAAGGGCCTATC | | |
| 17 | TCACTTTTCATGAGCAGGGATGC | FAM | Upstream of α2 |
| 18 | AAACAAACTTGGCTCTGGGTAGG | | gene |
| 19 | CTGCGGGCCTGGGCCGCACTGA | FAM | α2 gene |
| 20 | GCGGGCAGGAGGAACGGCTA | | |
| 21 | GCTGGTCGGAGCTACTTCCT | FAM | Upstream of α1 |
| 22 | TCATTTCCTGGGGGTCTGGC | | gene |
| 23 | GCTTTTTGCGTCCTGGTGTTGAT | FAM | |
| 24 | GCGAGTGCGAGCCGTGAG | | |
| 25 | CGGGCCTGGGCCCTCGGCCCCA | FAM | α1 gene |
| 26 | AGGGGCAAGAAGCATGGCC | | |
| 27 | GGGCTGTCAAGATCAGGCGT | FAM | Upstream of θ1 |
| 28 | GGTGACCTGGGGGTGAAAGT | | gene |
| 29 | TCACTGCCCTGAAGAAACACC | FAM | |
| 30 | CCGCGCCCGGCCATGTACATT | | |
| 31 | AGGTCAGGACGCGAGAGGAAG | FAM | Downstream of θ1 |
| 32 | TGGGCAGAGTCGGCCGTCCTCGCAG | | gene |
| 33 | CCTCTTGGATCCACGTTCTAGTTTC | FAM | |
| 34 | CTTCAGTGATGCTCCAGATGTAATCC | | |
| 35 | GATAGGGGTCCTCGGCCTG | FAM | |
| 36 | CTGTTTGGTGGGTGCTGTCG | | |
| 37 | CGGAGGCCAACCAGACTTGC | FAM | |
| 38 | AGTGGCTGAGACTTGTGTCCTG | | |
| 39 | CTTGCCAGGAACTTGTCCAGGGAGTCG | | α2: c.369C |
| 40 | CTTGTGTGTCAGGAACTTGTCCAGGGAGGACT | | α2: c.369C > G |
| 41 | CGGCTACCGAGGCTCCAGCGTA | | α2: c.427T |
| 42 | GTGTCGGCTACCGAGGCTCCAGCTGGA | | α2: c.427T > C |
| 43 | CTCACAGAAGCCAGGAACTTGTACA | | α2: c.377T |
| 44 | CTCACAGAAGCCAGGAACTTGTAAGG | | α2: c.377T > C |
| 45 | CAAAGGAGGATGTTTTTAGTAGC | N-(1- | Upstream |
| | | naphthyl)- | promoter region |
| | | ethylenediamine | of ß gene |
| | | (NED) | |
| 46 | GCTCTGCCCTGACTTTTCTG | | ß: c.−82C |
| 47 | GCTCTGCCCTGACTTTTAGTC | | ß: c.−82C > A |
| 48 | GATGGCTCTGCCCTGACTTGTA | | ß: c.−80T |
| 49 | GATGGCTCTGCCCTGACTTTGGT | | ß: c.−80T > C |
| 50 | AATAGATGGCTCTGCCCTGACTGTT | | ß: c.79A |
| 51 | TGGCTCTGCCCTGACTTAC | | ß: c.79A > G |
| 52 | GCAATAGATGGCTCTGCCCTGACGTT | | ß: c.78A |
| 53 | GCAATAGATGGCTCTGCCCTGACTGCT | | ß: c.78A > G |
| 54 | CACAGTTGTGTCAGAAGCAAAGGT | | ß: c.−50A |
| 55 | CAGTTGTGTCAGAAGCAAATTGA | | ß: c.−50A > C |
| 56 | CTCCTCAGGAGTCAGATGCAACA | | ß: c.2T |
| 57 | CTCCTCAGGAGTCAGATGCACACT | | ß: c.2T > G |
| 58 | ACGTTCACCTTGCCCCCCA | | ß: c.45_46 |
| 59 | ACGTTCACCTTGCCCCAACA | | ß: c.45_46insG |
| 60 | CCAACTTCATCCACGTTCAACT | | ß: c.52A |
| 61 | CCAACTTCATCCACGTTCACAAT | | ß: c.52A > T |
| 62 | ACCAACCTGCCCAGGGCATC | | ß: c.79G |
| 63 | ACCAACCTGCCCAGGGCCGTA | | ß: c.79G > A |
| 64 | GATACCAACCTGCCCATGGC | | ß: c.84_85 |
| 65 | GATACCAACCTGCCCAGGTGC | | ß: c.84_85insC |
| 66 | CTTGTAACCTTGATACCGAC | | ß: c.92 + 1G |
| 67 | CTTGTAACCTTGATACCACAC | | ß: c.92 + 1G > T |
| 68 | CTTGTAACCTTGATACCACTC | | ß: c.92 + 1G > A |
| 69 | TCCTTAAACCTGTCTTGTAACCTTGAGAC | | ß: c.92 + 5G |
| 70 | TCCTTAAACCTGTCTTGTAACCTTGATCG | | ß: c.92 + 5G > C |
| 71 | TCCTGAGACTTCCACACTGATG | NED | Exon 2 promoter |
| | | | region of ß gene |
| 72 | AAGAAAGTGCTCGGTGCCTTTCG | | ß: c.216_217 |
| 73 | AAGAAAGTGCTCGGTGCCTTGAAG | | ß: c.216_217insA |
| 74 | CCCTTGGACCCAGAGGTTCTGTG | | ß: c.130G |
| 75 | CCCCTTGGACCCAGAGGTTCTTATA | | ß: c.130G > T |
| 76 | TCTACCCTTGGACCCAGAGGTTCTTGGA | | ß: c.126_129 |
| 77 | TCTACCCTTGGACCCAGAGGGTG | | ß: c.126_129delCTTT |
| 78 | TTGGTCTATTTTCCCACCCTTATGC | | ß: c.94C |
| 79 | TTGGTCTATTTTCCCACCCTTAGTTG | | ß: c.94delC |
| 80 | TTGCACTGGTGGGGTGAATTCTT | NED | Exon 3 promoter |
| | | | region of ß gene |
| 81 | CAGTGATAATTTCTGGGTTAATGC | | ß: c.316-197 |
| 82 | ATAACAGTGATAATTTCTGGGTTAAGTTA | | ß: c.316-197C > T |
| 83 | CTTCTGCATCCTGTCGGCAA | FAM | ACTB |
| 84 | GACTGTCTCCCGGCTCTGCC | | |
| 85 | TAGGAAGTCACCAAAGCAGTACAGC | FAM | CFTR |
| 86 | TACTTGTACCAGCTCACTACCTA | | |
| 87 | ATGACACCTGCTTGCTCTC | FAM | RPP30 |
| 88 | AATCCATCCTATCTGGGAACATTAG | | |
| 89 | CCTTTATAGCAATAAATTCA | NED | RPP40 |
| 90 | ATGTAACGAAAATCTAGGGC | | |
| 91 | CCCTGGGCTCTGTAAAGAATAGTG | FAM | AMEL |
| 92 | ATCAGAGCTTAAACTGGGAAGCTG | | |
| 93 | GAATATTCCCGCTCTCCGGAG | FAM | SRY |
| 94 | GCTGGTGCTCCATTCTTGAGTG | | |

TABLE 2
Compositions of the reaction buffer

| Composition | Final Con. |
|---|---|
| 10X Taq Buffer (Thermo) | 1.5X |
| 25 mM MgCl₂ | 0.6 mM |
| dATP/dTTP/dGTP/dCTP | 0.5 mM each |
| Betaine | 2.0M |

TABLE 3
Concentration of the primer mixture

| Primer's SEQ ID NO. | Final Con. (μM) |
|---|---|
| 1 | 0.17 |
| 2 | 0.08 |
| 3 | 1.01 |
| 4 | 0.21 |
| 5 | 0.13 |
| 6 | 0.20 |
| 7 | 0.21 |
| 8 | 0.24 |
| 9 | 0.25 |
| 10 | 0.30 |
| 11 | 0.69 |
| 12 | 0.15 |
| 13 | 0.50 |
| 14 | 0.24 |
| 15 | 0.21 |
| 16 | 0.23 |
| 17 | 0.34 |
| 18 | 0.10 |
| 19 | 0.46 |
| 20 | 0.22 |
| 21 | 0.60 |
| 22 | 0.18 |
| 23 | 0.18 |
| 24 | 0.30 |
| 25 | 0.21 |
| 26 | 0.30 |
| 27 | 0.18 |
| 28 | 0.21 |
| 29 | 0.18 |
| 30 | 0.39 |
| 31 | 0.12 |
| 32 | 0.43 |
| 33 | 0.40 |
| 34 | 0.27 |
| 35 | 0.27 |
| 36 | 0.88 |
| 37 | 0.88 |
| 38 | 0.90 |
| 39 | 0.34 |
| 40 | 0.34 |
| 41 | 0.41 |
| 42 | 0.41 |
| 43 | 0.34 |
| 44 | 0.34 |
| 45 | 1.52 |
| 46 | 0.31 |
| 47 | 0.48 |
| 48 | 0.13 |
| 49 | 0.18 |
| 50 | 0.18 |
| 51 | 0.39 |
| 52 | 0.24 |
| 53 | 0.33 |
| 54 | 0.10 |
| 55 | 0.13 |
| 56 | 0.18 |
| 57 | 0.37 |
| 58 | 0.10 |
| 59 | 0.10 |
| 60 | 0.12 |
| 61 | 0.12 |
| 62 | 0.15 |
| 63 | 0.15 |
| 64 | 0.14 |
| 65 | 0.30 |
| 66 | 0.18 |
| 67 | 0.16 |
| 68 | 0.27 |
| 69 | 0.34 |
| 70 | 0.21 |
| 71 | 0.23 |
| 72 | 0.32 |
| 73 | 0.31 |
| 74 | 0.25 |
| 75 | 0.59 |
| 76 | 0.08 |
| 77 | 0.05 |
| 78 | 0.19 |
| 79 | 0.20 |
| 80 | 0.32 |
| 81 | 0.29 |
| 82 | 0.14 |
| 83 | 0.30 |
| 84 | 0.30 |
| 85 | 0.81 |
| 86 | 0.81 |
| 87 | 0.25 |
| 88 | 0.25 |
| 89 | 0.30 |
| 90 | 0.30 |
| 91 | 0.20 |
| 92 | 0.20 |
| 93 | 0.25 |
| 94 | 0.25 |

### Example 2: PCR Amplification

1. Preparation of DNA samples: nucleic acids were extracted from human whole blood samples with the aid of extraction kits (Cat. No: 04020001) provided by Biofast Biotechnology Co Ltd. (Xiamen, China). The extracted nucleic acids were subsequently amplified via PCR, with a DNA concentration from about 5-60 ng/μL, and OD₂₆₀ nm/OD₂₈₀ nm ratio of about 1.6-2.0.

2. Preparation of amplification reagent: the reaction buffer and primer mixtures of Example 1 were mixed thoroughly and centrifuged to bring down all the liquid. Amplification reagent was prepared in accordance with the conditions provided in Table 4.

TABLE 4
Amplification Reagent

| Component | Volume (μL) |
|---|---|
| α/β reaction buffer | 16 |
| α/β primer mixture | 5 |
| Hot start DNA polymerase | 0.2 |
| Total volume | 21.2 |

3. Preparation of PCR samples: In a PCR reaction vial, 21 μL of amplification reagent of Table 4 was mixed with 4 μL of nucleic acid sample, a normal control (i.e., DNA from normal healthy human subject), a positive control (i.e., DNA from α- or β-thalassemia human subject), or a blank control (i.e., Tris buffer, pH 8.5), the mixture in each PCR vial was then centrifuged.

### 4. PCR Amplification and Data Analysis

### (i) PCR Conditions

PCR reaction was performed with the conditions provided in Table 5 in total volume of 25 μL.

TABLE 5
PCR condition

| | | | | |
|---|---|---|---|---|
| 1 Hold | 95° | C. | 5 | min |
| 28 cycles | 95° | C. | 30 | sec |
| | 66° | C. | 40 | sec |
| | 72° | C. | 40 | sec |
| 1 Hold | 72° | C. | 45 | min |

| | | | |
|---|---|---|---|
| 1 Hold | 4° | C. | O/N |

### (ii) Fluorophore Marked Electrophoresis of the PCR Amplicons

The PCR amplicon (2 μL) was mixed with 1% GeneScan 500 LIZ Size Standard (10 μL) to give a mixture, which was denatured at 95° C. for 3 minutes, then analyzed via ABI3130, ABI3730, ABI3500Dx or ABI SeqStudio Genetic Analyzer.

### 5. Software

The data of PCR amplicons (i.e., fragment size) was analyzed by GeneMapper in accordance with user's guide provided by the software provider.

### 6. Calculation of Peak Ratio (R)

The peak areas of each target sites (i.e., the site where gene copy number intended to be identified) in the nucleic acid sample, the normal control, or the internal controls were independently divided by the sum of the peak area of internal controls, the value of the target site in the nucleic acid sample thus obtained was further divided by the value of its corresponding site in the normal control to produce the peak ratio (R) of each target site.

### 7. Data Interpretation

(1) The peak ratio (R) of internal control was found to be about 1.8-2.20, the relationship between peak ratio (R) and its corresponding gene copy number is shown in Table 6.

TABLE 6
Peak ratio and copy number

| Copy number | Peak ratio (R) |
|---|---|
| 0 | R ≤ 0.35 |
| 1 | 0.35 < R ≤ 1.42 |
| 2 | 1.42 < R ≤ 2.68 |
| 3 | R > 2.68 |

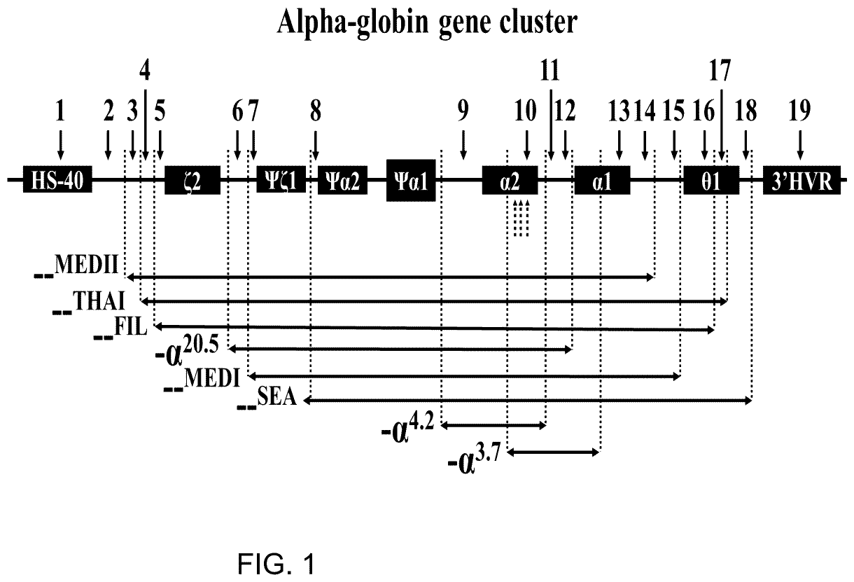

(2) The copy numbers of 19 target sites of α-globin gene cluster depicted in FIG. 1 were determined based on the criteria set forth in Table 7.

(3) The wild type β-globin with the copy number of 0 was indicated as homozygous deletion of β-globin gene; whereas the wild type β-globin with the copy 5 number of 1 was indicated as heterozygous deletion of β-globin gene.

(4) Determination of variants: In the case when only peaks are present in wild type loci, the sample was indicated as “negative”; in the case when only peaks are present in loci of variants, the sample was indicated as “homozygous mutant”; and in the case when peaks are present in both wild type loci and loci of variants, the sample was indicated as “heterozygous mutant”.

TABLE 7
Genotypes and gene copy number in each target site

| | | Target site # | | | | | | | | | | | | | | | | | | |
|---|---|---|---|---|---|---|---|---|---|---|---|---|---|---|---|---|---|---|---|---|
| Thalassemia | genotype | 1 | 2 | 3 | 4 | 5 | 6 | 7 | 8 | 9 | 10 | 11 | 12 | 13 | 14 | 15 | 16 | 17 | 18 | 19 |
| normal | αα/αα | 2 | 2 | 2 | 2 | 2 | 2 | 2 | 2 | 2 | 2 | 2 | 2 | 2 | 2 | 2 | 2 | 2 | 2 | 2 |
| heterozygous | -α3.7/αα | 2 | 2 | 2 | 2 | 2 | 2 | 2 | 2 | 2 | 1 | 1 | 1 | 2 | 2 | 2 | 2 | 2 | 2 | 2 |
| deletion | -α4.2/αα | 2 | 2 | 2 | 2 | 2 | 2 | 2 | 2 | 1 | 1 | 2 | 2 | 2 | 2 | 2 | 2 | 2 | 2 | 2 |
| | --SEA/αα | 2 | 2 | 2 | 2 | 2 | 2 | 2 | 1 | 1 | 1 | 1 | 1 | 1 | 1 | 1 | 1 | 1 | 1 | 2 |
| | --THAI/αα | 2 | 2 | 2 | 1 | 1 | 1 | 1 | 1 | 1 | 1 | 1 | 1 | 1 | 1 | 1 | 1 | 1 | 2 | 2 |
| | --FIL/αα | 2 | 2 | 2 | 2 | 1 | 1 | 1 | 1 | 1 | 1 | 1 | 1 | 1 | 1 | 1 | 1 | 2 | 2 | 2 |
| | --MED-I/αα | 2 | 2 | 2 | 2 | 2 | 2 | 1 | 1 | 1 | 1 | 1 | 1 | 1 | 1 | 1 | 2 | 2 | 2 | 2 |
| | --MED-II/αα | 2 | 2 | 1 | 1 | 1 | 1 | 1 | 1 | 1 | 1 | 1 | 1 | 1 | 1 | 2 | 2 | 2 | 2 | 2 |
| | -α20.5/αα | 2 | 2 | 2 | 2 | 2 | 1 | 1 | 1 | 1 | 1 | 1 | 1 | 2 | 2 | 2 | 2 | 2 | 2 | 2 |
| homozygous | -α3.7/-α3.7 | 2 | 2 | 2 | 2 | 2 | 2 | 2 | 2 | 2 | 0 | 0 | 0 | 2 | 2 | 2 | 2 | 2 | 2 | 2 |
| deletion | -α4.2/-α4.2 | 2 | 2 | 2 | 2 | 2 | 2 | 2 | 2 | 0 | 0 | 2 | 2 | 2 | 2 | 2 | 2 | 2 | 2 | 2 |
| | --SEA/--SEA | 2 | 2 | 2 | 2 | 2 | 2 | 2 | 0 | 0 | 0 | 0 | 0 | 0 | 0 | 0 | 0 | 0 | 0 | 2 |
| | --THAI/--THAI | 2 | 2 | 2 | 0 | 0 | 0 | 0 | 0 | 0 | 0 | 0 | 0 | 0 | 0 | 0 | 0 | 0 | 2 | 2 |
| | --FIL/--FIL | 2 | 2 | 2 | 2 | 0 | 0 | 0 | 0 | 0 | 0 | 0 | 0 | 0 | 0 | 0 | 0 | 2 | 2 | 2 |
| | --MED-I/--MED-I | 2 | 2 | 2 | 2 | 2 | 2 | 0 | 0 | 0 | 0 | 0 | 0 | 0 | 0 | 0 | 2 | 2 | 2 | 2 |
| | --MED-II/--MED-II | 2 | 2 | 0 | 0 | 0 | 0 | 0 | 0 | 0 | 0 | 0 | 0 | 0 | 0 | 2 | 2 | 2 | 2 | 2 |
| | -α20.5/-α20.52 | 2 | 2 | 2 | 2 | 2 | 0 | 0 | 0 | 0 | 0 | 0 | 0 | 2 | 2 | 2 | 2 | 2 | 2 | 2 |
| Gene | ααα anti-3.7/αα | 2 | 2 | 2 | 2 | 2 | 2 | 2 | 2 | 2 | 3 | 3 | 3 | 2 | 2 | 2 | 2 | 2 | 2 | 2 |
| duplication | ααα anti4.2/αα | 2 | 2 | 2 | 2 | 2 | 2 | 2 | 2 | 3 | 3 | 2 | 2 | 2 | 2 | 2 | 2 | 2 | 2 | 2 |
| HS-40 heterozygous | delHS-40 αα/αα | 1 | 2 | 2 | 2 | 2 | 2 | 2 | 2 | 2 | 2 | 2 | 2 | 2 | 2 | 2 | 2 | 2 | 2 | 2 |
| deletion | | | | | | | | | | | | | | | | | | | | |
| HS-40 homozygous | delHS-40/delHS-40 | 0 | 2 | 2 | 2 | 2 | 2 | 2 | 2 | 2 | 2 | 2 | 2 | 2 | 2 | 2 | 2 | 2 | 2 | 2 |
| deletion | αα/αα | | | | | | | | | | | | | | | | | | | |

### Example 3: Characterization of the Present Primer Kit

### 3.1 Detection Precision of the Present Primers/Methods

A total of 16 reference samples were prepared for detecting samples that were in high (60 ng/μL), medium (25 ng/μL), or low (15 ng/μL) concentrations. The 16 reference samples listed in Table 8 included 6 deletion-type α-thalassemia samples, 1 α-triplex sample, 2 non-deletional form of α-thalassemia samples, and 7 non-deletion form of β-thalassemia samples. The detection was repeated 3 times for 16 samples respectively at high, medium and low concentrations, and three batches of samples were tested. The gene copy numbers and peaks in all detected loci were found to comply with relevant criteria provided in Tables 6 and 7.

TABLE 8
Reference samples

| | | | α₂ | β |
|---|---|---|---|---|
| Reference samples | Name | variants | non-deletional | non-deletional |
| Deletional form of | THAL-PC01 | −−^{SEA}/αα | None | None |
| α-thalassemia | THAL-PC02 | −α^{3.7}/αα | None | None |
| | THAL-PC03 | −α^{4.2}/αα | None | None |
| | THAL-PC04 | −−^{THAI}/αα | None | None |
| | THAL-PC05 | −−^{FIL}/αα | None | None |
| | THAL-PC06 | −α^{3.7}/−α^{3.7} | None | None |
| α-globin gene | THAL-PC07 | ααα^{anti-3.7}/αα | None | None |
| triplication | | | | |
| non-deletional form of | THAL-PC08 | α^{QS}α/αα | c.377T > C | None |
| α-thalassemia | THAL-PC09 | α^{CS}α/α^{WS}α | c.427T > C, | None |
| | | | c.369C > G | |
| non-deletional form of | THAL-PC10 | αα/αα | None | c.−82C > A, |
| β-thalassemia | | | | c.2T > G, |
| | | | | c.92 + 1G > A |
| | THAL-PC11 | αα/αα | None | c.−80T > C, |
| | | | | c.45_46insG, |
| | | | | c.130G > T |
| | THAL-PC12 | αα/αα | None | c.79A > G, |
| | | | | c.92 + 1G > T, |
| | | | | c.126_129delCTTT |
| | THAL-PC13 | αα/αα | None | c.78A > G, |
| | | | | c.52A > T, |
| | | | | c.94delC |
| | THAL-PC14 | αα/αα | None | c.−50A > C, |
| | | | | c.84_85insC |
| | THAL-PC15 | αα/αα | None | c.79G > A, |
| | | | | c.92 + 5G > C |
| | THAL-PC16 | αα/αα | None | c.216_217insA, |
| | | | | c.316-197C > T |

### 3.2 Test of Repeatability

Four repetitive samples listed in Table 9 were used in this example, and the test for samples at each concentration was repeated 10 times, using three batches of amplification agents. The gene copy numbers and peaks of the target loci were found to be accurate and comply with relevant criteria.

TABLE 9
Reference samples of deletion form α-thalassemia, and
non-deletional form of α-and β-thalassemia

| | | | α₂ non- | β non- |
|---|---|---|---|---|
| Sample | Name | Type | deletional | deletional |
| 4 | THAL-LPC01 | −α^{3.7}/αα | None | None |
| repetitive | THAL-LPC02 | α^{CS}α/αα | c.427T > C | None |
| reference | THAL-LPC03 | αα/αα | None | None |
| samples | THAL-LPC04 | αα/αα | None | c.216_217insA, |
| | | | | c.316 − 197C > T |

### 3.3 Limit of Detection

A total of 16 reference samples in Table 10 were provided for determining the detection limits. Each sample was tested at the concentrations of 20 ng/μL or 15 ng/μL, and each test was repeated 20 times. A total of three batches of samples were tested, and results were found to comply with relevant criteria.

TABLE 10
Reference samples of deletion form α-thalassemia, and
non-deletional form of α-and β-thalassemia

| Samples | Name | type | α₂ non-deletional | β non-deletional |
|---|---|---|---|---|
| reference | THAL-LOD01 | −−^{SEA}/αα | None | None |
| samples for | THAL-LOD02 | −α^{3.7}/αα | None | None |
| determining | THAL-LOD03 | −α^{4.2}/αα | None | None |
| detection | THAL-LOD04 | −−^{THAI}/αα | None | None |
| limit | THAL-LOD05 | −−^{FIL}/αα | None | None |
| | THAL-LOD06 | −α^{3.7}/−α^{3.7} | None | None |
| | THAL-LOD07 | ααα^{anti-3.7}/αα | None | None |
| | THAL-LOD08 | α^{QS}α/αα | c.377T > C | None |
| | THAL-LOD09 | α^{CS}α/α^{WS}α | c.427T > C, | None |
| | | | c.369C > G | |
| | THAL-LOD10 | αα/αα | None | c.−82C > A, c.2T > G, |
| | | | | c.92 + 1G > A |
| | THAL-LOD11 | αα/αα | None | c.−80T > C, c.45_46insG, |
| | | | | c.130G > T |
| | THAL-LOD12 | αα/αα | None | c.79A > G, c.92 + 1G > T, |
| | | | | c.126_129delCTTT |
| | THAL-LOD13 | αα/αα | None | c.78A > G, c.52A > T, |
| | | | | c.94delC |
| | THAL-LOD14 | αα/αα | None | c.−50A > C, c.84_85insC |
| | THAL-LOD15 | αα/αα | None | c.79G > A, c.92 + 5G > C |
| | THAL-LOD16 | αα/αα | None | c.216_217insA, c.316 − |
| | | | | 197C > T |

### Example 4: Identifying Thalassemia Genotype Via the Present Primers/Methods

### 4.1 Deletional Form of α-Thalassemia and α-Globin Gene Triplication

One hundred and fifty EDTA-treated whole blood samples were collected under written consent for the identification of deletional form of α-thalassemia and α-globin gene triplication via use of the present methods/primers. To verify data consistency, the methods developed by MRC-Holland Inc were also performed.

Human genomic nucleic acids from EDTA-treated whole blood samples were extracted by use of nucleic acid extraction kits (Cat. No: 04020001) provided by Biofast Biotechnology Co Ltd. (Xiamen, China). The concentration and the purity of the extracted nucleic acids were determined by microplate reader. The 150 samples independently had a concentration of 15-50 ng/L, and OD₂₆₀ nm/OD₂₈₀ nm ratio of about 1.6-2.0.

PCR amplification was conducted in accordance with procedures described in Example 2. The PCR amplicons were analyzed and the results are summarized in Table 11. The data in Table 11 was found to be consistent with copy number criteria for α-globin gene target loci in the total of 150 samples, among them, 6 samples had α-thalassemia, 97 samples were α-thalassemia carriers, and 47 samples were normal healthy subjects.

TABLE 11
Identification results of 150 whole blood samples

| | | Type | |
|---|---|---|---|
| Sample # | Type | MLPA* | The present invention |
| 1 | α-thalassemia | --^{SEA}/-α^{3.7} | --^{SEA}/-α^{3.7} |
| 2 | α-thalassemia | --^{SEA}/--^{SEA} | --^{SEA}/--^{SEA} |
| 3 | α-thalassemia | --^{SEA}/--^{SEA} | --^{SEA}/--^{SEA} |
| 4 | α-thalassemia | --^{SEA}/--^{SEA} | --^{SEA}/--^{SEA} |
| 5 | α-thalassemia | --^{SEA}/-α^{4.2} | --^{SEA}/-α^{4.2} |
| 6 | α-thalassemia | --^{SEA}/--^{SEA} | --^{SEA}/--^{SEA} |
| 7 | Carrier of α-thalassemia | --^{SEA}/αα | --^{SEA}/αα |
| 8 | Carrier of α-thalassemia | --^{SEA}/αα | --^{SEA}/αα |
| 9 | Carrier of α-thalassemia | --^{SEA}/αα | --^{SEA}/αα |
| 10 | Carrier of α-thalassemia | --^{SEA}/αα | --^{SEA}/αα |
| 11 | Carrier of α-thalassemia | --^{SEA}/αα | --^{SEA}/αα |
| 12 | Carrier of α-thalassemia | --^{SEA}/αα | --^{SEA}/αα |
| 13 | Carrier of α-thalassemia | -α^{3.7}/αα | -α^{3.7}/αα |
| 14 | Carrier of α-thalassemia | -α^{3.7}/-α^{3.7} | -α^{3.7}/-α^{3.7} |
| 15 | Carrier of α-thalassemia | -α^{3.7}/αα | -α^{3.7}/αα |
| 16 | Carrier of α-thalassemia | -α^{4.2}/αα | -α^{4.2}/αα |
| 17 | Carrier of α-thalassemia | --^{SEA}/αα | --^{SEA}/αα |
| 18 | Carrier of α-thalassemia | --^{SEA}/αα | --^{SEA}/αα |
| 19 | Carrier of α-thalassemia | --^{SEA}/αα | --^{SEA}/αα |
| 20 | Carrier of α-thalassemia | -α^{3.7}/αα | -α^{3.7}/αα |
| 21 | Carrier of α-thalassemia | --^{FIL}/αα | --^{FIL}/αα |
| 22 | Carrier of α-thalassemia | --^{SEA}/αα | --^{SEA}/αα |
| 23 | Carrier of α-thalassemia | --^{SEA}/αα | --^{SEA}/αα |
| 24 | Carrier of α-thalassemia | --^{SEA}/αα | --^{SEA}/αα |
| 25 | Carrier of α-thalassemia | --^{SEA}/αα | --^{SEA}/αα |
| 26 | Carrier of α-thalassemia | --^{THAI}/αα | --^{THAI}/αα |
| 27 | Carrier of α-thalassemia | --^{SEA}/αα | --^{SEA}/αα |
| 28 | Carrier of α-thalassemia | --^{SEA}/αα | --^{SEA}/αα |
| 29 | Carrier of α-thalassemia | --^{SEA}/αα | --^{SEA}/αα |
| 30 | Carrier of α-thalassemia | --^{SEA}/αα | --^{SEA}/αα |
| 31 | Carrier of α-thalassemia | --^{SEA}/αα | --^{SEA}/αα |
| 32 | Carrier of α-thalassemia | --^{SEA}/αα | --^{SEA}/αα |
| 33 | Carrier of α-thalassemia | --^{SEA}/αα | --^{SEA}/αα |
| 34 | Carrier of α-thalassemia | --^{SEA}/αα | --^{SEA}/αα |
| 35 | Carrier of α-thalassemia | --^{SEA}/αα | --^{SEA}/αα |
| 36 | Carrier of α-thalassemia | --^{SEA}/αα | --^{SEA}/αα |
| 37 | Carrier of α-thalassemia | --^{SEA}/αα | --^{SEA}/αα |
| 38 | Carrier of α-thalassemia | --^{FIL}/αα | --^{FIL}/αα |
| 39 | Carrier of α-thalassemia | --^{SEA}/αα | --^{SEA}/αα |
| 40 | Carrier of α-thalassemia | --^{SEA}/αα | --^{SEA}/αα |
| 41 | Carrier of α-thalassemia | --^{SEA}/αα | --^{SEA}/αα |
| 42 | Carrier of α-thalassemia | --^{SEA}/αα | --^{SEA}/αα |
| 43 | Carrier of α-thalassemia | -α^{3.7}/αα | -α^{3.7}/αα |
| 44 | Carrier of α-thalassemia | -α^{3.7}/αα | -α^{3.7}/αα |
| 45 | Carrier of α-thalassemia | --^{FIL}/αα | --^{FIL}/αα |
| 46 | Carrier of α-thalassemia | --^{SEA}/αα | --^{SEA}/αα |
| 47 | Carrier of α-thalassemia | --^{SEA}/αα | --^{SEA}/αα |
| 48 | Carrier of α-thalassemia | --^{SEA}/αα | --^{SEA}/αα |
| 49 | Carrier of α-thalassemia | --^{SEA}/αα | --^{SEA}/αα |
| 50 | Carrier of α-thalassemia | -α^{3.7}/αα | -α^{3.7}/αα |
| 51 | Carrier of α-thalassemia | --^{SEA}/αα | --^{SEA}/αα |
| 52 | Carrier of α-thalassemia | --^{SEA}/αα | --^{SEA}/αα |
| 53 | Carrier of α-thalassemia | --^{SEA}/αα | --^{SEA}/αα |
| 54 | Carrier of α-thalassemia | --^{SEA}/αα | --^{SEA}/αα |
| 55 | Carrier of α-thalassemia | --^{SEA}/αα | --^{SEA}/αα |
| 56 | Carrier of α-thalassemia | --^{FIL}/αα | --^{FIL}/αα |
| 57 | Carrier of α-thalassemia | -α^{3.7}/αα | -α^{3.7}/αα |
| 58 | Carrier of α-thalassemia | --^{SEA}/αα | --^{SEA}/αα |
| 59 | Carrier of α-thalassemia | --^{SEA}/αα | --^{SEA}/αα |
| 60 | Carrier of α-thalassemia | --^{SEA}/αα | --^{SEA}/αα |
| 61 | Carrier of α-thalassemia | --^{SEA}/αα | --^{SEA}/αα |
| 62 | Carrier of α-thalassemia | --^{SEA}/αα | --^{SEA}/αα |
| 63 | Carrier of α-thalassemia | --^{SEA}/αα | --^{SEA}/αα |
| 64 | Carrier of α-thalassemia | --^{SEA}/αα | --^{SEA}/αα |
| 65 | Carrier of α-thalassemia | -α^{3.7}/-α^{3.7} | -α^{3.7}/-α^{3.7} |
| 66 | Carrier of α-thalassemia | --^{SEA}/αα | --^{SEA}/αα |
| 67 | Carrier of α-thalassemia | --^{SEA}/αα | --^{SEA}/αα |
| 68 | Carrier of α-thalassemia | --^{SEA}/αα | --^{SEA}/αα |
| 69 | Carrier of α-thalassemia | -α^{3.7}/-α^{4.2} | -α^{3.7}/-α^{4.2} |
| 70 | Carrier of α-thalassemia | -a3.7/aa | -a3.7/aa |
| 71 | Carrier of α-thalassemia | --SEA/αα | --SEA/αα |
| 72 | Carrier of α-thalassemia | --SEA/αα | --SEA/αα |
| 73 | Carrier of α-thalassemia | --SEA/αα | --SEA/αα |
| 74 | Carrier of α-thalassemia | -α^{4.2}/αα | -α^{4.2}/αα |
| 75 | Carrier of α-thalassemia | --^{SEA}/αα | --^{SEA}/αα |
| 76 | Carrier of α-thalassemia | --^{SEA}/αα | --^{SEA}/αα |
| 77 | Carrier of α-thalassemia | -α^{3.7}/-α^{3.7} | -α^{3.7}/-α^{3.7} |
| 78 | Carrier of α-thalassemia | ααα^{anti-3.7}/--^{SEA} | ααα^{anti-3.7}/--^{SEA} |
| 79 | Carrier of α-thalassemia | --^{SEA}/αα | --^{SEA}/αα |
| 80 | Carrier of α-thalassemia | --^{SEA}/αα | --^{SEA}/αα |
| 81 | Carrier of α-thalassemia | --^{SEA}/αα | --^{SEA}/αα |
| 82 | Carrier of α-thalassemia | --^{SEA}/αα | --^{SEA}/αα |
| 83 | Carrier of α-thalassemia | --^{SEA}/αα | --^{SEA}/αα |
| 84 | Carrier of α-thalassemia | --^{SEA}/αα | --^{SEA}/αα |
| 85 | Carrier of α-thalassemia | --^{SEA}/αα | --^{SEA}/αα |
| 86 | Carrier of α-thalassemia | --^{SEA}/αα | --^{SEA}/αα |
| 87 | Carrier of α-thalassemia | --^{FIL}/αα | --^{FIL}/αα |
| 88 | Carrier of α-thalassemia | -α^{3.7}/αα | -α^{3.7}/αα |
| 89 | Carrier of α-thalassemia | --^{SEA}/αα | --^{SEA}/αα |
| 90 | Carrier of α-thalassemia | --^{FIL}/αα | --^{FIL}/αα |
| 91 | Carrier of α-thalassemia | --^{SEA}/αα | --^{SEA}/αα |
| 92 | Carrier of α-thalassemia | --^{SEA}/αα | --^{SEA}/αα |
| 93 | Carrier of α-thalassemia | --^{SEA}/αα | --^{SEA}/αα |
| 94 | Carrier of α-thalassemia | -α^{3.7}/αα | -α^{3.7}/αα |
| 95 | Carrier of α-thalassemia | --^{SEA}/αα | --^{SEA}/αα |
| 96 | Carrier of α-thalassemia | --^{SEA}/αα | --^{SEA}/αα |
| 97 | Carrier of α-thalassemia | --^{SEA}/αα | --^{SEA}/αα |
| 98 | Carrier of α-thalassemia | --^{FIL}/αα | --^{FIL}/αα |
| 99 | Carrier of α-thalassemia | --^{SEA}/αα | --^{SEA}/αα |
| 100 | Carrier of α-thalassemia | --^{FIL}/αα | --^{FIL}/αα |
| 101 | Carrier of α-thalassemia | --^{SEA}/αα | --^{SEA}/αα |
| 102 | Carrier of α-thalassemia | --^{SEA}/αα | --^{SEA}/αα |
| 103 | Carrier of α-thalassemia | --^{SEA}/αα | --^{SEA}/αα |
| 104 | α-thalassemia (normal) | αα/αα | αα/αα |
| 105 | α-thalassemia (normal) | αα/αα | αα/αα |
| 106 | α-thalassemia (normal) | αα/αα | αα/αα |
| 107 | α-thalassemia (normal) | αα/αα | αα/αα |
| 108 | α-thalassemia (normal) | αα/αα | αα/αα |
| 109 | α-thalassemia (normal) | αα/αα | αα/αα |
| 110 | α-thalassemia (normal) | αα/αα | αα/αα |
| 111 | α-thalassemia (normal) | αα/αα | αα/αα |
| 112 | α-thalassemia (normal) | αα/αα | αα/αα |
| 113 | α-thalassemia (normal) | αα/αα | αα/αα |
| 114 | α-thalassemia (normal) | αα/αα | αα/αα |
| 115 | α-thalassemia (normal) | αα/αα | αα/αα |
| 116 | α-thalassemia (normal) | αα/αα | αα/αα |
| 117 | α-thalassemia (normal) | ααα^{anti-3.7}/αα | ααα^{anti-3.7}/αα |
| 118 | α-thalassemia (normal) | αα/αα | αα/αα |
| 119 | α-thalassemia (normal) | αα/αα | αα/αα |
| 120 | α-thalassemia (normal) | αα/αα | αα/αα |
| 121 | α-thalassemia (normal) | αα/αα | αα/αα |
| 122 | α-thalassemia (normal) | αα/αα | αα/αα |
| 123 | α-thalassemia (normal) | αα/αα | αα/αα |
| 124 | α-thalassemia (normal) | αα/αα | αα/αα |
| 125 | α-thalassemia (normal) | αα/αα | αα/αα |
| 126 | α-thalassemia (normal) | αα/αα | αα/αα |
| 127 | α-thalassemia (normal) | αα/αα | αα/αα |
| 128 | α-thalassemia (normal) | αα/αα | αα/αα |
| 129 | α-thalassemia (normal) | αα/αα | αα/αα |
| 130 | α-thalassemia (normal) | αα/αα | αα/αα |
| 131 | α-thalassemia (normal) | αα/αα | αα/αα |
| 132 | α-thalassemia (normal) | αα/αα | αα/αα |
| 133 | α-thalassemia (normal) | αα/αα | αα/αα |
| 134 | α-thalassemia (normal) | αα/αα | αα/αα |
| 135 | α-thalassemia (normal) | αα/αα | αα/αα |
| 136 | α-thalassemia (normal) | αα/αα | αα/αα |
| 137 | α-thalassemia (normal) | αα/αα | αα/αα |
| 138 | α-thalassemia (normal) | αα/αα | αα/αα |
| 139 | α-thalassemia (normal) | αα/αα | αα/αα |
| 140 | α-thalassemia (normal) | αα/αα | αα/αα |
| 141 | α-thalassemia (normal) | αα/αα | αα/αα |
| 142 | α-thalassemia (normal) | αα/αα | αα/αα |
| 143 | α-thalassemia (normal) | αα/αα | αα/αα |
| 144 | α-thalassemia (normal) | αα/αα | αα/αα |
| 145 | α-thalassemia (normal) | αα/αα | αα/αα |
| 146 | α-thalassemia (normal) | αα/αα | αα/αα |
| 147 | α-thalassemia (normal) | αα/αα | αα/αα |
| 148 | α-thalassemia (normal) | αα/αα | αα/αα |
| 149 | α-thalassemia (normal) | αα/αα | αα/αα |
| 150 | α-thalassemia (normal) | αα/αα | αα/αα |

*MLPA: Multiplex Ligation-dependent Probe Amplification, served as the normal control group in the present disclosure.

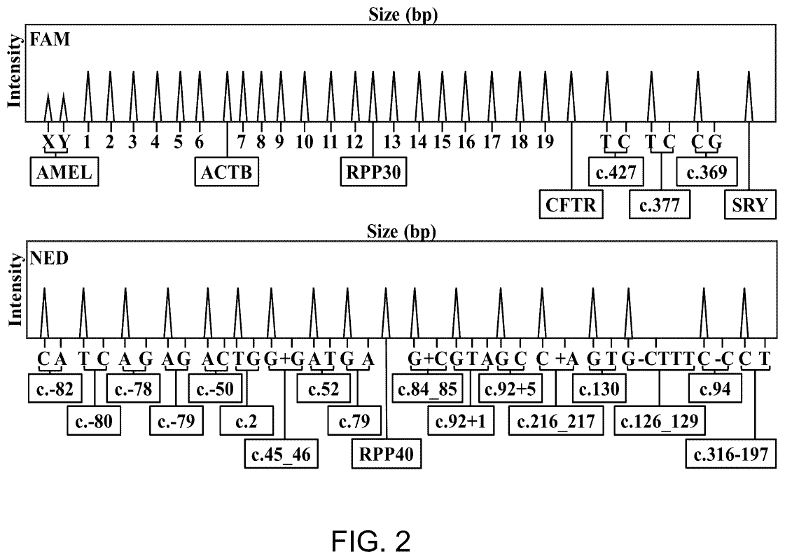

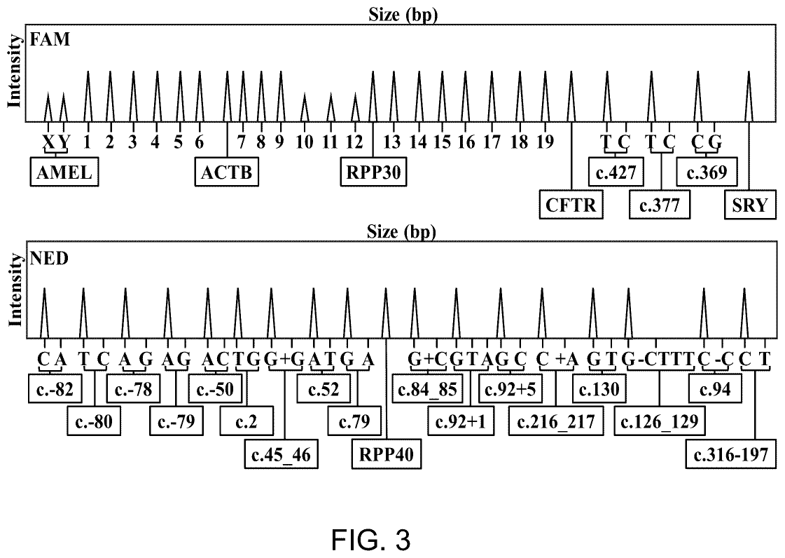

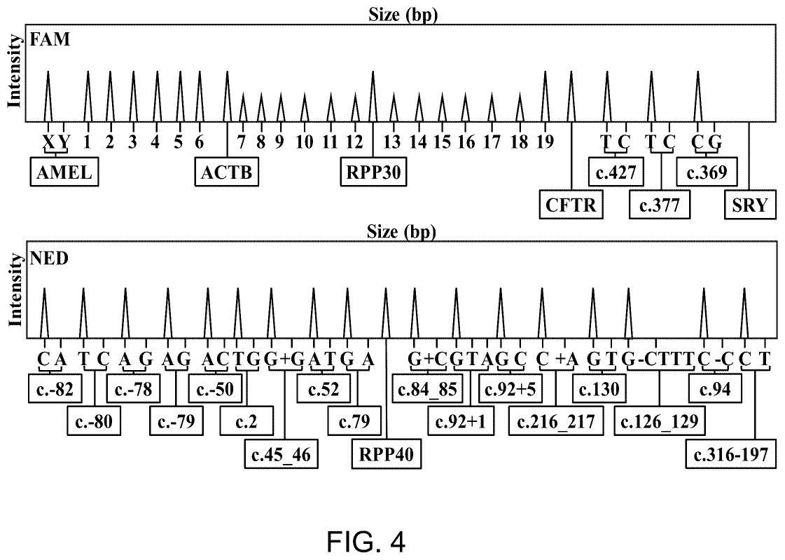

According to Table 11 and FIGS. 1 to 4, in which FIGS. 2 to 4 respectively illustrates results of sample #104, 13, and 7, it was found that primers of the present disclosure could accurately determine the gene copy numbers of human α-globin genes, differentiate subjects having α-thalassemia from subjects carrying α-thalassemia and normal healthy subjects, furthermore, the sex of each sample.

### 4.2 Non-Deletional Forms of α- or β-Thalassemia

The primers/methods of the present disclosure were used to identify non-deletional forms of α- or β-thalassemia in a total of 21 EDTA-treated whole blood samples. Further, to verify data consistency, Sanger sequencing amplification method was also used to identify non-deletional forms of α- or β-thalassemia in the 21 samples.

Human genomic nucleic acids from EDTA-treated whole blood samples were extracted by use of nucleic acid extraction kits (Cat. No: 04020001) provided by Biofast Biotechnology Co Ltd. (Xiamen, China). The concentration and the purity of the extracted nucleic acids were determined by microplate reader. The 21 samples independently had a concentration of 15-50 ng/μL, and OD₂₆₀ nm/OD₂₈₀ nm ratio of about 1.6-2.0.

PCR amplification was conducted in accordance with procedures described in Example 2. The PCR amplicons were analyzed and the results are summarized in Table 12. According to Table 12, 2 samples were identified as α-thalassemia carriers of α^{WS}α/αα (α2:c.369C>G), 1 sample as α^{QS}α/αα (α2:c.377T>C), 1 sample as α^{CS}α/αα (α2:c.427T>C), 1 sample as β-thalassemia of β^{IVS-II-654}/β^{IVS-II-654} (β:c.316-197C>T), 6 samples as β-thalassemia carriers of β^{CD41-42}/BN (β:c.126_129delCTTT), 7 samples as β^{IVS-II-654}/β^{N} (β:c.316-197C>T), and 3 samples as β^{CD26}/β^{N} (β:c.79G>A). The identification was consistent with that obtained by Sanger sequencing amplification method. Taken together, the present primers/methods could identify subjects of α-thalassemia carriers and subjects of β-thalassemia.

TABLE 12
Identification results of 25 whole blood samples

| | | Detection of point | | | | | |
|---|---|---|---|---|---|---|---|
| | | mutation in α2 gene | | | | | |
| | | | The | | | Detection of β-globin gene | |
| Sample | | Sanger | present | Sample | | mutation and small deletion | |
| # | Type | method | method | # | Type | Sanger method | The present method |
| 1 | Carrier of α- | c.369C > G | c.369C > G | 5 | Carrier of β- | c.316-197C > T | c.316-197C > T |
| | thalassemia | | | | thalassemia | | |
| 2 | Carrier of α- | c.377T > C | c.377T > C | 6 | Carrier of β- | c.126_129delCTTT | c.126_129delCTTT |
| | thalassemia | | | | thalassemia | | |
| 3 | Carrier of α- | c.369C > G | c.369C > G | 7 | Carrier of β- | c.126_129delCTTT | c.126_129delCTTT |
| | thalassemia | | | | thalassemia | | |
| 4 | Carrier of α- | c.427T > C | c.427T > C | 8 | Carrier of β- | c.316-197C > T | c.316-197C > T |
| | thalassemia | | | | thalassemia | | |
| | | | | 9 | Carrier of β- | c.316-197C > T | c.316-197C > T |
| | | | | | thalassemia | | |
| | | | | 10 | Carrier of β- | c.316-197C > T | c.316-197C > T |
| | | | | | thalassemia | | |
| | | | | 11 | Carrier of β- | c.316-197C > T | c.316-197C > T |
| | | | | | thalassemia | | |
| | | | | 12 | Carrier of β- | c.79G > A | c.79G > A |
| | | | | | thalassemia | | |
| | | | | 13 | Carrier of β- | c.316-197C > T | c.316-197C > T |
| | | | | | thalassemia | | |
| | | | | 14 | Carrier of β- | c.126_129delCTTT | c.126 129delCTTT |
| | | | | | thalassemia | | |
| | | | | 15 | Carrier of β- | c.79G > A | c.79G > A |
| | | | | | thalassemia | | |
| | | | | 16 | Carrier of β- | c.79G > A | c.79G > A |
| | | | | | thalassemia | | |
| | | | | 17 | Carrier of β- | c.126_129delCTTT | c.126_129delCTTT |
| | | | | | thalassemia | | |
| | | | | 18 | Carrier of β- | c.126_129delCTTT | c.126_129delCTTT |
| | | | | | thalassemia | | |
| | | | | 19 | Carrier of β- | c.316-197C > T | c.316-197C > T |
| | | | | | thalassemia | | |
| | | | | 20 | Carrier of β- | c.316-197C > T | c.316-197C > T |
| | | | | | thalassemia | | |
| | | | | 21 | Carrier of β- | c.126_129delCTTT | c.126_129delCTTT |
| | | | | | thalassemia | | |

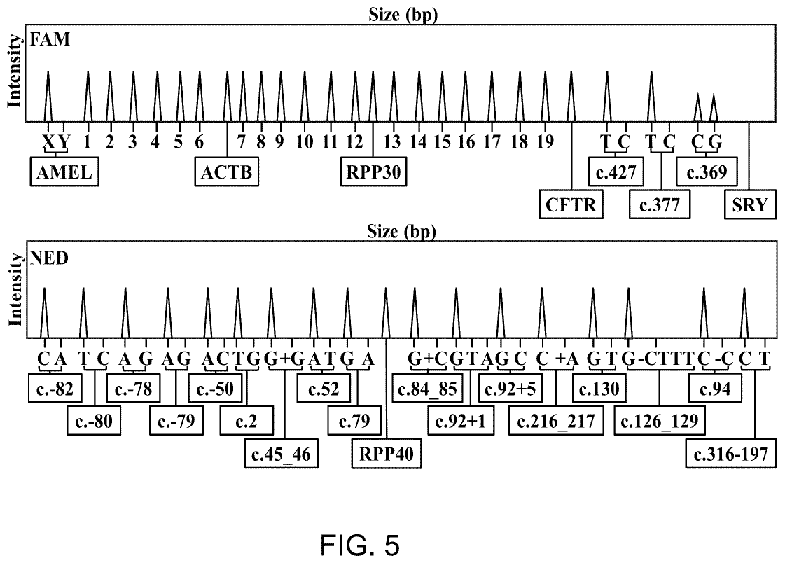

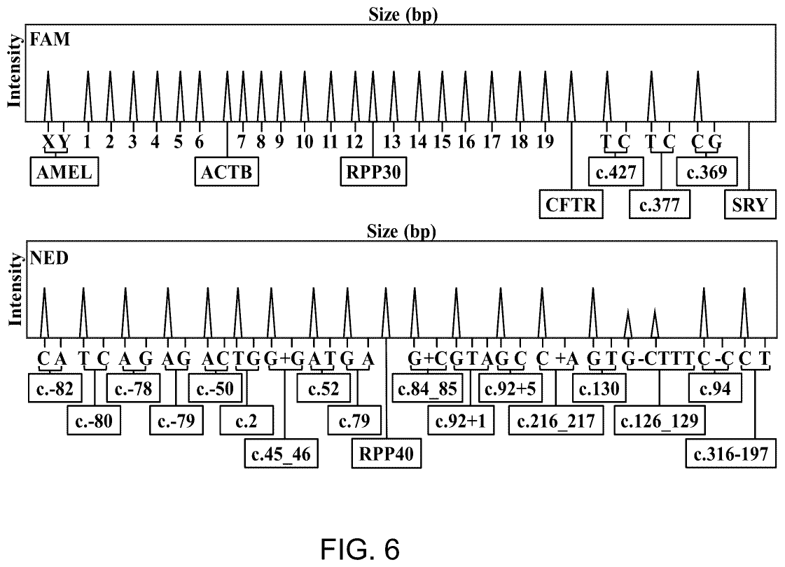

According to Table 12, and FIGS. 5 and 6, the present primers/methods could accurately determine variants in α2-globin gene and β-globin gene, thereby may successfully differentiate thalassemia subjects and its carriers from healthy normal subjects.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A kit for determining a genotype of a subject having thalassemia comprising:
a first group of primers respectively having the nucleic acid sequences of SEQ ID Nos: 1-38 for targeting 19 target sites in ζ2, ψζ1, Ψα2, Ψα1, α2, α1 and θ1 genes in α-globin gene cluster;
a second group of primers respectively having the nucleic acid sequences of SEQ ID Nos: 39-44 for targeting α^{WS}α, α^{QS}α, and α^{CS}α variants in the α2-globin gene;
a third group of primers respectively having the nucleic acid sequences of SEQ ID Nos: 45-82 for targeting β⁻³², β⁻³⁰, β⁻²⁹, β⁻²⁸, β^{Cap+1}, β^{IntM}, β^{CD14-15}, β^{CD17}, β^{CD26}, β^{CD27/28}, β^{IVS-I-1(G>T)}, β^{TVS-I-1(G>A)}, β^{IVS-I-5}, β^{CD71-72}, β^{CD43}, β^{CD41-42}, β^{CD31}, and β^{IVS-II-654} variants in β-globin gene;
a fourth group of primers respectively having the nucleic acid sequence of SEQ ID Nos: 83-90 for targeting a reference gene; and
a fifth group of primers respectively having the nucleic acid sequence of SEQ ID Nos: 91-94 for targeting a chromosomal gene.

2. The kit of claim 1, wherein the reference gene is selected from the group consisting of glyceraldehyde-3-phosphate dehydrogenase (GAPDH), actin beta (ACTB), cystic fibrosis transmembrane conductance regulator (CFTR), hypoxanthine-guanine phosphoribosyltransferase (HPRT), ribonuclease P protein subunit p30 (RPP30), ribonuclease P protein subunit p40 (RPP40), and a combination thereof.

3. The kit of claim 1, wherein the chromosomal gene is selected from the group consisting of amelogenin (AMEL), zinc finger protein, X-linked (ZFX), zinc finger protein, Y-linked (ZFY), TATA-box binding protein associated factor 9 (TAF9), sex-determining region Y protein (SRY), and a combination thereof.

4. The kit of claim 1, wherein the primer is labeled with a fluorescent molecule selected from the group consisting of carboxyfluorescein (FAM), 2′-chloro-7′-phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX).

5. The kit of claim 1, further comprising,
an amplification reagent comprising a hot start DNA polymerase and deoxynucleotide triphosphates; and
a normal control, a positive control, and a blank control;
wherein,
the normal control is a genomic DNA of a healthy subject;
the positive control is a genomic DNA of a subject of α-thalassemia or β-thalassemia;
the blank control is a buffer solution.

6. A method for determining a genotype of a subject having thalassemia comprising:
(a) mixing a nucleic acid sample with a primer mixture and an amplification reagent to produce a reaction mixture, wherein, the primer mixture consists of the first, second, third, fourth and fifth groups of primers of claim 1, and the nucleic acid sample is a genomic DNA isolated from the subject, a healthy subject, or an α-thalassemia or β-thalassemia subject;
(b) subjecting the reaction mixture to a polymerase chain reaction (PCR) to produce amplicons;
(c) subjecting the amplicons to capillary electrophoresis to separate the amplicons from one another thereby generating a plurality of peaks independently corresponds to one separated amplicon;
(d) determining the peak area of each separated amplicon of step (c), in which each separated amplicon corresponds to a gene targeting by the first, second, third, fourth or fifth groups of primers;
(e) calculating a peak ratio (R) of the gene targeted by the first, second or third groups of primers in step (d);
(f) determining a copy number of the gene targeted by the first, second or third groups of primers based on the calculated R of step (e); and
(g) determining the genotype of the subject based on the determined copy number in step (f);
wherein,
in step (e), the R is calculated by,
(i) dividing the peak area of each separated amplicon corresponds to the gene targeted by the first, second or third groups of primers with the sum of the peak area of an internal control to generate a first value; and
(ii) dividing the first value of step (i) with a second value derived from the same gene in the normal control; and
in step (f), the copy number of the gene is 0 when R is ≤0.35, the copy number of the gene is 1 when 0.35<R≤1.42, the copy number of the gene is 2 when 1.42<R≤2.68 or the copy number of the gene is 3 when R>2.68.

7. The method of claim 6, wherein, the amplification reagent comprises a hot start DNA polymerase and deoxynucleotide triphosphates.

8. The method of claim 7, wherein at least one primer is labeled with a fluorescent molecule selected from the group consisting of FAM, VIC, HEX, JOE, TMR, NED, and ROX.

9. The method of claim 7, wherein in step (b), the PCR is performed under the conditions of:
(1) 95° C. for 5 minutes;
(2) 28 cycles of the following: 95° C. for 30 seconds, 66° C. for 40 seconds, and 72° C. for 40 seconds;
(3) 72° C. for 45 minutes; and
(4) 4° C.

10. The method of claim 6, wherein the internal control is one or more reference genes independently selected from the group consisting of glyceraldehyde-3-phosphate dehydrogenase (GAPDH), actin beta (ACTB), cystic fibrosis transmembrane conductance regulator (CFTR), hypoxanthine-guanine phosphoribosyltransferase (HPRT), ribonuclease P protein subunit p30 (RPP30), ribonuclease P protein subunit p40 (RPP40), and a combination thereof.

11. The method of claim 10, wherein the internal control consists of ACTB, CFTR, RPP30 and RPP40.

12. The method of claim 6, further comprising repeating the method by use of a blank control, which is a buffer solution.

13. The method of claim 11, wherein in step (g),
the subject has heterozygous deletion form of thalassemia with −α3.7/αα genotype when the gene copy number is 1 for each target sites Nos. 10-12, and the gene copy number is 2 for each of the rest target sites;
the subject has heterozygous deletion form of thalassemia with −α4.2/αα genotype when the gene copy number is 1 for each target site Nos. 9-10, and the gene copy number is 2 for each of the rest target sites;
the subject has heterozygous deletion form of thalassemia with −^{SEA}/αα genotype when the gene copy number is 1 for each target site Nos.: 8-18, and the gene copy number is 2 for each of the rest target sites;
the subject has heterozygous deletion form of thalassemia with −^{THA1}/αα genotype when the gene copy number is 1 for each target site Nos: 4-17 and the gene copy number is 2 for each of the rest target sites;
the subject is determined to have heterozygous deletion form of thalassemia with −^{FIL}/αα genotype when the gene copy number is 1 for each target site Nos: 5-16, and the gene copy number is 2 for each of the rest target sites;
the subject has heterozygous deletion form of thalassemia with −^{MED-I}/αα genotype when the gene copy number is 1 for each target site Nos: 7-15, and the gene copy number is 2 for each of the rest target sites;
the subject has heterozygous deletion form of thalassemia with −^{MED-I}/αα genotype when the gene copy number is 1 for each target site Nos: 3-14, and the gene copy number is 2 for each of the rest target sites;
the subject has heterozygous deletion form of thalassemia with −α20.5/αα genotype when the gene copy number is 1 for each target site Nos: 6-12 and the gene copy number is 2 for each of the rest target sites;
the subject has homozygous deletion form of thalassemia with −α3.7/−α3.7 genotype when the gene copy number is 0 for each target site Nos: 10-12, and the gene copy number is 2 for each of the rest target sites;
the subject has homozygous deletion form of thalassemia with −α4.2/−α4.2 genotype when the gene copy number is 0 for each target site Nos: 9-10, and the gene copy number is 2 for each of the rest target sites;
the subject has homozygous deletion form of thalassemia with −^{SEA}/−^{SEA} genotype when the gene copy number is 0 for each target site Nos: 7-18, and the gene copy number is 2 for each of the rest target sites;
the subject has homozygous deletion form of thalassemia with −^{THA1}/−^{THA1} genotype when the gene copy number is 0 for each target site Nos: 4-17, and the gene copy number is 2 for each of the rest target sites;
the subject has homozygous deletion form of thalassemia with −^{FIL}/−^{FIL} genotype when the gene copy number is 0 for each target site Nos: 5-16, and the gene copy number is 2 for each of the rest target sites;
the subject has homozygous deletion form of thalassemia with −^{MED-I}/−^{MED-I} genotype when the gene copy number is 0 for each target site Nos: 7-15, and the gene copy number is 2 for each of the rest target sites;
the subject has homozygous deletion form of thalassemia with −^{MED-II}/−^{MED-II} genotype when the gene copy number is 0 for each target site Nos: 3-14, and the gene copy number is 2 for each of the rest target sites;
the subject has homozygous deletion form of thalassemia with −^{20.5}/−α^{20.5} genotype when the gene copy number is 0 for each target site Nos: 6-12, and the gene copy number is 2 for each of the rest of target sites;
the subject has duplication form of thalassemia with ααα anti-3.7/αα genotype when the gene copy number is 3 for each target site Nos: 10-12, and the gene copy number is 2 for each of the rest target sites;
the subject has duplication form of thalassemia with ααα anti-4.2/αα genotype when the gene copy number is 3 for each target site Nos: 9-10, and the gene copy number is 2 for each of the rest target sites;
the subject has HS-40 heterozygous deletion form of thalassemia with del HS-40 αα/αα genotype when the gene copy number is 1 for the target site No: 1, and the gene copy number is 2 for each the rest target sites;
the subject has HS-40 homozygous deletion form of thalassemia with del HS-40/del HS-40 αα/αα genotype when the gene copy number is 0 for the target site No: 1, and the gene copy number is 2 for each of the rest target sites;
the subject has homozygous deletion of β-globin gene when the gene copy number for β-globin is 0; or
the subject has heterozygous deletion of β-globin gene when the gene copy number of β-globin is 1.
